SEQUENCE LISTING (17 sequences)

SEQ ID NO: 1 - RNA, 20 nt, synthetic construct
  source 1..20: mol_type = other RNA
  misc_feature 1..20: note = CRISPR target sequence
atccaccgttgagagctggg

SEQ ID NO: 2 - DNA, 500 bp, synthetic construct
  misc_feature 1..500: note = FOXP3 5' homology arm
  source 1..500: mol_type = other DNA
agaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatgtgccccccag

SEQ ID NO: 3 - DNA, 500 bp, synthetic construct
  source 1..500: mol_type = other DNA
  misc_feature 1..500: note = FOXP3 3' homology arm
ctctcaacggtggatgcccacgcccggacccctgtgctgcaggtgcaccccctggagagcccagccatgatcagcctcacaccacccaccaccgccactggggtcttctccctcaaggcccggcctggcctcccacctggtaacacctcagcccgtaccccatggcttcacagaacccccaagtccccagatccttggctgtgagcagtgtaggctattctgaattgcagtactctgggggtcaaaggtgtcaggtctcagaggcttggaaactccaccctccaaaaaacgtcaggtgcagaaccttaaagatgcagaatgtcaaaatcacaaaaccacagagctttacaaagctagtcaaaatgtcagcacctgcgaatggccgtctttaagcttctctgccagaagcctgggactttggggacagcagagccccctgggagtcagggttttcgaggctcaggagggtgggaagctcaaaatgagaggccttgtgggcc

SEQ ID NO: 4 - DNA, 166 bp, synthetic construct
  source 1..166: mol_type = other DNA
  misc_feature 1..166: note = FOXP3 exon 1
gcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaag

SEQ ID NO: 5 - DNA, 231 bp, synthetic construct
  source 1..231: mol_type = other DNA
  misc_feature 1..231: note = FOXP3 exon 2
cctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgca

SEQ ID NO: 6 - DNA, 106 bp, synthetic construct
  source 1..106: mol_type = other DNA
  misc_feature 1..106: note = FOXP3 exon 3
gctgcccacactgcccctagtcatggtggcaccctccggggcacggctgggccccttgccccacttacaggcactcctccaggacaggccacatttcatgcaccag

SEQ ID NO: 7 - DNA, 139 bp, synthetic construct
  source 1..139: mol_type = other DNA
  misc_feature 1..139: note = FOXP3 exon 4
ctctcaacggtggatgcccacgcccggacccctgtgctgcaggtgcaccccctggagagcccagccatgatcagcctcacaccacccaccaccgccactggggtcttctccctcaaggcccggcctggcctcccacctg

SEQ ID NO: 8 - DNA, 513 bp, synthetic construct
  source 1..513: mol_type = other DNA
  misc_feature 1..513: note = PGK promoter
aattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 9 - DNA, 68 bp, synthetic construct
  source 1..68: mol_type = other DNA
  misc_feature 1..68: note = Mini CMV Promoter
tatgtcgaggtggcgtgtacggtgggaggcctatataagcagagctcgtttagtgaaccgtcagatcg

SEQ ID NO: 10 - DNA, 1256 bp, synthetic construct
  source 1..1256: mol_type = other DNA
  misc_feature 1..1256: note = Ubiquitin C promoter
ccgcggctaaggaacgcgggccgcccacccgctccgggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttagtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggagggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 11 - DNA, 825 bp, synthetic construct
  source 1..825: mol_type = other DNA
  misc_feature 1..825: note = Delta LNGFR
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 12 - DNA, 222 bp, synthetic construct
  source 1..222: mol_type = other DNA
  misc_feature 1..222: note = SV40 poly A
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 13 - DNA, 141 bp, synthetic construct
  source 1..141: mol_type = other DNA
  misc_feature 1..141: note = left AA V2 ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 14 - DNA, 141 bp, synthetic construct
  source 1..141: mol_type = other DNA
  misc_feature 1..141: note = right AA V2 ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 5840 bp, synthetic construct
  source 1..5840: mol_type = other DNA
  misc_feature 1..5840: note = FOXP3.PKGprom.NGF.I TR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatgtgccccccagtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgctatcacctcttgaaggctatgtaggccacaagacccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcggggcactcctcgcacacggtgttctgcttgtcctggcaggagaacacgaggcccgagcccgcctcgcacacgcggcacgcctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatggcgcggccggtggcacctgcccccatggtggcgaattccgatctgacggttcactaaacgagctctgcttatataggcctcccaccgtacacgccacctcgacataaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccttagacgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctggtaacacctcagcccgtaccccatggcttcacagaacccccaagtccccagatccttggctgtgagcagtgtaggctattctgaattgcagtactctgggggtcaaaggtgtcaggtctcagaggcttggaaactccaccctccaaaaaacgtcaggtgcagaaccttaaagatgcagaatgtcaaaatcacaaaaccacagagctttacaaagctagtcaaaatgtcagcacctgcgaatggccgtctttaagcttctctgccagaagcctgggactttggggacagcagagccccctgggagtcagggttttcgaggctcaggagggtgggaagctcaaaatgagaggccttgtgggccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaag

SEQ ID NO: 16 - DNA, 6801 bp, synthetic construct
  source 1..6801: mol_type = other DNA
  misc_feature 1..6801: note = FOXP3.UBCprom.NGFR.ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatgtgccccccagtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgctatcacctcttgaaggctatgtaggccacaagacccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcggggcactcctcgcacacggtgttctgcttgtcctggcaggagaacacgaggcccgagcccgcctcgcacacgcggcacgcctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatggcgcggccggtggcacctgcccccatggtggcgaattccgatctgacggttcactaaacgagctctgcttatataggcctcccaccgtacacgccacctcgacataccgcggctaaggaacgcgggccgcccacccgctccgggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttagtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggagggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctggtaacacctcagcccgtaccccatggcttcacagaacccccaagtccccagatccttggctgtgagcagtgtaggctattctgaattgcagtactctgggggtcaaaggtgtcaggtctcagaggcttggaaactccaccctccaaaaaacgtcaggtgcagaaccttaaagatgcagaatgtcaaaatcacaaaaccacagagctttacaaagctagtcaaaatgtcagcacctgcgaatggccgtctttaagcttctctgccagaagcctgggactttggggacagcagagccccctgggagtcagggttttcgaggctcaggagggtgggaagctcaaaatgagaggccttgtgggccgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - protein, 5 aa, synthetic construct
  REGION 1..5: note = consensus sequence
RXKRR

(19) United States
(12) Patent Application Publication
(10) Pub. No.: US 2024/0350628 A1
(43) Pub. Date: Oct. 24, 2024

(54) METHODS FOR CREATING REGULATORY T CELLS (TREGS) USING GENOME ENGINEERING

(71) Applicant: Lung Biotechnology PBC, Silver Spring, MD (US)

(72) Inventors: Colin EXLINE, Silver Spring, MD (US); Kurt WONG, Silver Spring, MD (US); Sean STEVENS, Silver Spring, MD (US); Huizhu LIU, Silver Spring, MD (US)

(21) Appl. No.: 18/611,471

(22) Filed: Mar. 20, 2024

Related U.S. Application Data
(60) Provisional application No. 63/491,481, filed on Mar. 21, 2023.

(51) Int. Cl. A61K 39/00; C07K 14/47; C12N 5/0783; C12N 9/22; C12N 15/11; C12N 15/90

(52) U.S. Cl. CPC A61K 39/4621; A61K 39/4611; A61K 39/4637; A61K 39/46434; C07K 14/4702; C12N 5/0637; C12N 9/22; C12N 15/11; C12N 15/907; A61K 2239/26; C12N 2310/20; C12N 2510/00; C12N 2800/80

(57) ABSTRACT

Provided are methods, polynucleotides and compositions for the generation of engineered Treg cells. The methods, polynucleotides and compositions enable the reprogramming of hematopoietic cells into Treg cells by constitutive or regulated expression of FOXP3 in engineered cells such that the engineered Treg cells can suppress the activation and proliferation of responder T cells.

### CROSS-REFERENCE TO RELATED APPLICATIONS AND INCORPORATION BY REFERENCE

This application claims the priority benefit of U.S. Provisional Application No. 63/491,481, filed on Mar. 21, 2023, which is herein incorporated by reference in its entirety.

### REFERENCE TO SEQUENCE LISTING SUBMITTED ELECTRONICALLY

The content of the electronically submitted ST.26 sequence listing in XML format (Name 5064_0020002_SequenceListing_ST26.xml; Size: 39,719 bytes; and Date of Creation: Mar. 18, 2023) filed with the application is incorporated herein by reference in its entirety.

### FIELD OF DISCLOSURE

The present disclosure pertains to regulatory T cells. Some aspects of the disclosure are directed to a polynucleotide sequence comprising a bidirectional promoter operably linked to a FOXP3 exon and a selectable marker protein.

### BACKGROUND

Regulatory T cells are a subset of T cells that act to limit the scope and reaction of other T cell subsets. They play crucial roles in protection from off target damages during an infection and function to protect against aberrant reactions targeting healthy tissues. Due to their low abundance, regulatory T cells have been difficult to produce. Retroviral vectors delivering a forkhead box protein 3 (FOXP3) complementary DNA (cDNA) expression cassette have been used to confer Treg-like properties to conventional T cells in humans and mice. However, the stability of a Treg phenotype is dependent on the strength of the promoter and the maintenance of high expression of FOXP3, and randomly integrating a vector results in a heterogeneous cell product, with variable numbers of viral integration sites scattered nonspecifically across the genome, and occasionally gene silencing.

Therefore, there is a need for methods and polynucleotide constructs that provide stable, high-level FOXP3 expression in target cells to produce Treg cells.

### BRIEF SUMMARY

Provided are methods, polynucleotides, and compositions for creating regulatory T cells by gene editing to engineer and reprogram normal T cells into a regulatory phenotype capable of persisting within the body at an increased number. The methods and materials provide FOXP3 donor constructs for editing of the genomic FOXP3 site such that the FOXP3 gene is modified to comprise the FOXP3 donor construct. The FOXP3 donor construct comprises a heterologous bidirectional promoter driving expression of a selectable marker protein and FOXP3 in gene-edited cells. Advantageously, the methods and materials increase the number of regulatory T cells without the need for large-scale cellular amplification, as such amplification can lead to T cell exhaustion. Furthermore, by placing the FOXP3 gene under the control of a heterologous promoter, it is no longer controlled by cellular mechanisms that prevent normal T cells from becoming regulatory T cells. The methods and materials described herein can be used to treat auto-immunity and prevent and/or treat rejection associated with allo- and xeno-transplantation.

In some aspects, provided is a method of making a polynucleotide for expression of FOXP3, the method comprising: (i) providing a first nucleotide sequence, wherein the first nucleotide sequence comprises a coding strand and a targeted locus, the coding strand comprising: one or more regulatory elements and a FOXP3 gene; and the targeted locus comprising an intron sequence of the FOXP3 gene; (ii) providing a second nucleic acid sequence; (iii) providing a nuclease; and (iv) performing a gene editing process on the first nucleotide sequence, which edits said intron sequence, and inserts the second nucleic acid into the targeted locus, wherein insertion of the second nucleic acid results in expression of FOXP3.

In some aspects, the completion of the editing process results in a knock in process for insertion of the second nucleotide sequence in the targeted locus, wherein the second nucleotide sequence comprises a heterologous promoter operably linked to a polynucleotide comprising at least one FOXP3 exon or a portion thereof.

In some aspects, the second nucleotide sequence further comprises a polynucleotide encoding a selectable marker protein. In some aspects, the selectable marker protein is a cell surface protein. In some aspects, the selectable marker protein is a truncated, low-affinity nerve growth factor receptor protein.

In some aspects, the heterologous promoter is a bidirectional promoter and controls transcription of the polynucleotide comprising the at least one FOXP3 exon or portion thereof and the polynucleotide encoding the selectable marker protein in opposite directions.

In some aspects, the second nucleotide sequence comprises exons 1, 2, and 3 of FOXP3 and the targeted locus is at an intron between exon 2 and exon 3 of the FOXP3 gene of the first nucleotide sequence. In some aspects, the second nucleotide sequence comprises exons 1, 2, 3, and 4 of FOXP3 and the targeted locus is at an intron between exon 3 and exon 4 of the FOXP3 gene of the first nucleotide sequence.

In some aspects, the nuclease is a Cas9, a zinc finger nuclease, a TALEN, an engineered meganuclease, or a restriction endonuclease.

Further provided is a polynucleotide for FOXP3 expression made by the method described herein. In some aspects, the polynucleotide comprises: a coding strand that comprises a heterologous promoter operably linked to a polynucleotide encoding a selectable marker protein and located between an intronic regulatory T cell (Treg)-specific demethylation region (TSDR) of a FOXP3 gene and an exon of the FOXP3 gene.

In some aspects, the heterologous promoter of the polynucleotide is located between the TSDR of the FOXP3 gene and a first exon of the FOXP3 gene.

In some aspects, the heterologous promoter is bidirectional and promotes the transcription of the polynucleotide encoding the selectable marker protein in the direction of the TSDR and promotes the transcription of the first exon of the FOXP3 gene in the opposite direction.

In some aspects, the selectable marker protein of the polynucleotide is a cell surface protein. In some aspects, the selectable marker protein of the polynucleotide is a truncated, low-affinity nerve growth factor receptor protein.

Also provided is a system comprising a polynucleotide comprising a bidirectional heterologous promoter operably linked to polynucleotide encoding a selectable marker protein and at least a first exon of a FOXP3 gene, and a nuclease.

In some aspects, the polynucleotide of the system further comprises a 5′ arm and a 3′ arm that each are homologous to a portion of an intron sequence of a FOXP3 gene.

In some aspects, the 5′ arm and the 3′ arm of the polynucleotide are each homologous to a portion of an intron that is located between a second and a third exon of a FOXP3 gene and wherein the polynucleotide comprises a first, second, and third FOXP3 exon.

In some aspects, the 5′ arm and the 3′ arm of the polynucleotide are each homologous to a portion of an intron that is located between a third and a fourth exon of the FOXP3 gene and wherein the polynucleotide comprises a first, second, third, and fourth FOXP3 exon.

In some aspects, the nuclease of the system is a Cas9, a zinc finger nuclease, a TALEN nuclease, an engineered meganuclease, or a restriction endonuclease.

In some aspects, the selectable marker protein of the system is a cell surface protein. In some aspects, the cell surface protein of the system is a truncated, low-affinity nerve growth factor receptor protein.

Further provided is a method of inducing FOXP3 expression in a cell, the method comprising administering a system as described herein. In some aspects, the method further comprises culturing the cell and measuring cell surface expression of the truncated, low-affinity nerve growth factor receptor on the cell, wherein the level of expression of the truncated, low-affinity nerve growth factor receptor is indicative of the level of FOXP3 expression in the cell.

Provided is also a method of suppressing T cell activation using an engineered regulatory T cell, the method comprising: inducing FOXP3 expression in a T cell using the method described herein to prepare an engineered regulatory T cell; co-incubating the engineered regulatory T cell with a non-engineered T cell and a xenograft cell; wherein the activation of the non-engineered T cell by the xenograft cell is suppressed.

The methods provided herein can be used to suppress an immune reaction in a subject receiving non-HLA matched (i.e., “unmatched”) donor cells. For example, an engineered regulatory T cell prepared according to a method described herein can be administered to a subject prior to, concomitant with, or after the administration of non-HLA matched donor cells. Using the methods described herein, donor cell-reactive T cells of the subject are engineered to express FOXP3 and, upon administration to the subject, inhibit T cell activation by the unmatched donor cells.

For example, provided is a method of suppressing T cell activation using an engineered regulatory T cell, the method comprising: contacting a T cell with an unmatched donor cell; isolating a donor cell-reactive T cell; inducing FOXP3 expression in the donor-cell reactive T cell using the method described herein to prepare an engineered donor-cell reactive T cell; and co-incubating the engineered donor-cell reactive T cell with a non-engineered T cell and an unmatched donor cell; wherein the activation of the non-engineered T cell by the unmatched donor cell is suppressed.

In some aspects, the engineered regulatory T cell is a human cell. In some aspects, the unmatched donor cell is a human cell.

Further provided is a vector comprising a polynucleotide comprising a bidirectional heterologous promoter operably linked to polynucleotide encoding a selectable marker protein and further operably linked to a polynucleotide comprising at least a first exon of a FOXP3 gene.

In some aspects, the polynucleotide of the vector further comprises a 5′ arm and a 3′ arm that each are homologous to a portion of an intron and/or exon sequence of a FOXP3 gene.

In some aspects, the selectable marker protein of the vector is a cell surface protein. In some aspects, the cell surface protein is a truncated, low-affinity nerve growth factor receptor protein.

Also provided is a cell comprising a polynucleotide described herein.

Additionally, provided is a composition comprising a system described herein, a vector described herein, or a cell described herein and a delivery vehicle.

In some aspects, the delivery vehicle of the composition is a viral vector or a lipid.

In some aspects, the lipid of the composition is comprised within a lipid vehicle.

In some aspects, the viral vector of the composition is selected from the group consisting of adeno-associated virus, adenovirus, retrovirus, orthomyxovirus, paramyxovirus, papovavirus, picornavirus, lentivirus, herpes simplex virus, vaccinia virus, pox virus, and alphavirus.

Also provided is a pharmaceutical composition comprising a system as described herein, a vector as described herein, or a cell as described herein and a pharmaceutically acceptable carrier or excipient.

Provided further is a method for inducing, regulating, or enhancing the expression of a FOXP3 gene in a subject comprising (a) administering a system described herein to the subject.

And, provided is a method of controlling inflammation in a subject in need thereof comprising operatively coupling a heterologous promoter to a FOXP3 gene, or a portion thereof, and, in the opposite direction, operatively coupling the heterologous promoter to a polynucleotide encoding a selectable marker protein, wherein the heterologous promoter promotes transcription of the FOXP3 gene, or portion thereof, and the polynucleotide of the selectable marker protein, wherein the expression of the FOXP3 gene controls inflammation in the subject.

In some aspects, the method further comprises administering to the subject a therapeutically effective amount of a vector as described herein; a cell as described herein; a composition as described herein; or a pharmaceutical composition as described herein.

Further provided is a method of making a genetically engineered cell, the method comprising: providing a cell, wherein the cell comprises a first nucleic acid comprising at least one targeted locus; providing a Cas9 protein or a second nucleic acid encoding a Cas9 protein; introducing the Cas9 protein or the second nucleic acid into the cell; introducing a third nucleic acid encoding at least one CRISPR guide sequence, wherein the at least one CRISPR guide sequence is configured to hybridize to the at least one targeted locus; and introducing a fourth nucleic acid into the cell, wherein the fourth nucleic acid comprises a bidirectional heterologous promoter operably linked to a nucleic acid sequence encoding a selectable marker protein and at least a first exon of a FOXP3 gene, wherein the Cas9 protein and CRISPR guide sequence induce introduction of the fourth nucleic acid into the first nucleic acid at the at least one targeted locus and thereby genetically engineer the cell.

In some aspects, the targeted locus of the method is an intron of a FOXP3 gene.

In some aspects, the selectable marker protein used in the method is a cell surface protein. In some aspects, the cell surface protein used in the method is a truncated nerve growth factor receptor protein.

Also provided is a method of reducing xenotransplant rejection in a patient, the method comprising administering to a patient prior to, or at the same time as, transplanting a xenotransplant in the patient, a therapeutically effective amount of cells prepared according to the methods described herein, wherein the administered cells reduce xenotransplant rejection.

And provided is a method of increasing immunological tolerance to a xenotransplant in a patient, the method comprising administering to a patient prior to, or at the same time as, transplanting a xenotransplant in the patient, a therapeutically effective amount of cells prepared according to the methods described herein, wherein the administered cells increase immunological tolerance to the xenotransplant.

Further provided is a method of reducing allotransplant rejection in a patient, the method comprising administering to a patient prior to, or at the same time as, transplanting an allotransplant in the patient, a therapeutically effective amount of cells prepared according to the methods described herein, wherein the administered cells reduce allotransplant rejection.

And provided is a method of increasing immunological tolerance to a allotransplant in a patient, the method comprising administering to a patient prior to, or at the same time as, transplanting a allotransplant in the patient, a therapeutically effective amount of cells prepared according to the methods described herein, wherein the administered cells increase immunological tolerance to the allotransplant.

### BRIEF DESCRIPTION OF THE DRAWINGS/FIGURES

FIG. 1 shows a schematic of a normal human FOXP3 gene (top); a schematic of a normal human FOXP3 gene, a FOXP3 donor construct, and a nuclease (oval symbol) (middle); and a schematic of the final edited human FOXP3 gene (bottom). The “selectable gene” has been written backwards to indicate its transcription in an opposite direction relative to the FOXP3 gene.

FIG. 2 shows a schematic of a FOXP3 donor construct comprising a human ubiquitin C promoter, LNGFR marker gene and flanking FOXP3 gene portions (top) and a schematic of a FOXP3 donor construct comprising a PGK promoter, delta LNGFR marker gene and flanking FOXP3 homology arms (bottom).

FIG. 3A shows a schematic of the generation of gene-edited Treg cells from activated CD4+ cells using AAV6 for FOXP3 donor construct expression and Cas9 RNP electroporation. FIG. 3B shows flow cytometry scans of mock treated, AAV donor only treated, FOXP3 targeting CRISPR/Cas9 only treated and FOXP3 targeting CRISPR/Cas9 and AAV donor construct treated CD4+ cells depicting FOXP3 expression on the y-axes and selectable gene expression on the x-axes.

FIG. 4A shows a schematic of the generation of gene-edited Treg cells from activated CD4+ cells using AAV6 for FOXP3 donor construct expression and Cas9 RNP electroporation followed by culture of edited cells for 1-week before assaying for the edited cell marker. FIG. 4B shows a flow cytometry scan identifying edited Treg cells expressing FOXP3 and the selectable marker.

FIG. 5A shows cell proliferation flow cytometry scans of unstimulated responder T cells and responder T cells stimulated with a T cell stimulation reagent (anti-CD3, anti-CD28, anti-CD2) and co-cultured with engineered Tregs at different Treg to T responder cell ratios. FIG. 5B shows a histogram of replication indices of unstimulated and stimulated responder T cells following co-cultures with engineered Tregs at different Treg to T responder cell ratios.

FIG. 6A shows a graph of mean fluorescence intensities for expression of CD154 and CD49d on unstimulated and stimulated responder T cells co-cultured with engineered Tregs at different Treg to responder T cell ratios. FIG. 6B shows a graph of mean fluorescence intensities of the expression of CD45RA and CCR7 on unstimulated and stimulated responder T cells co-cultured with engineered Tregs at different Treg to responder T cell ratios. FIG. 6C shows a graph of mean fluorescence intensities of the expression of PD-1 and CD25 on unstimulated and stimulated responder T cells co-cultured with engineered Tregs at different Treg to responder T cell ratios.

FIG. 7A shows levels of cytokine IL-2 in the supernatant of unstimulated and stimulated responder T cells co-cultured with engineered Tregs at different Treg to responder T cell ratios. FIG. 7B shows levels of cytokine IL-10 in the supernatant of unstimulated and stimulated responder T cells co-cultured with engineered Tregs at different Treg to responder T cell ratios. FIG. 7C shows levels of cytokine IL-4 in the supernatant of unstimulated and stimulated responder T cells co-cultured with engineered Tregs at different Treg to responder T cell ratios.

FIG. 8A shows cell proliferation flow cytometry scans of unstimulated responder T cells and responder T cells stimulated with porcine cells, then co-cultured with engineered Tregs at different Treg to T responder cell ratios. FIG. 8B shows a histogram of T cell activation in unstimulated responder T cells and responder T cells stimulated with porcine cells, then co-cultured with engineered Tregs at different Treg to T responder cell ratios. FIG. 8C shows a histogram of IFN-7 expression in unstimulated responder T cells and responder T cells stimulated with porcine cells, then co-cultured with engineered Tregs at different Treg to T responder cell ratios.

FIG. 9 shows cell proliferation flow cytometry scans of unstimulated responder T cells and responder T cells stimulated with unmatched human donor cells (primary human Pulmonary Endothelial Cells) in the absence and presence of bulk engineered Tregs and directed Tregs at different Treg to T responder cell ratios.

FIG. 10A shows a histogram of T cell activation of responder T cells stimulated with unmatched human donor cells, then cultured in the absence of Tregs, in the presence of bulk Tregs or in the presence of allo-donor specific Tregs. FIG. 10B shows a histogram of the percentage of maximal activation of responder T cells stimulated with unmatched human donor cells, then cultured in the presence of bulk Tregs or in the presence of allo-donor specific Tregs.

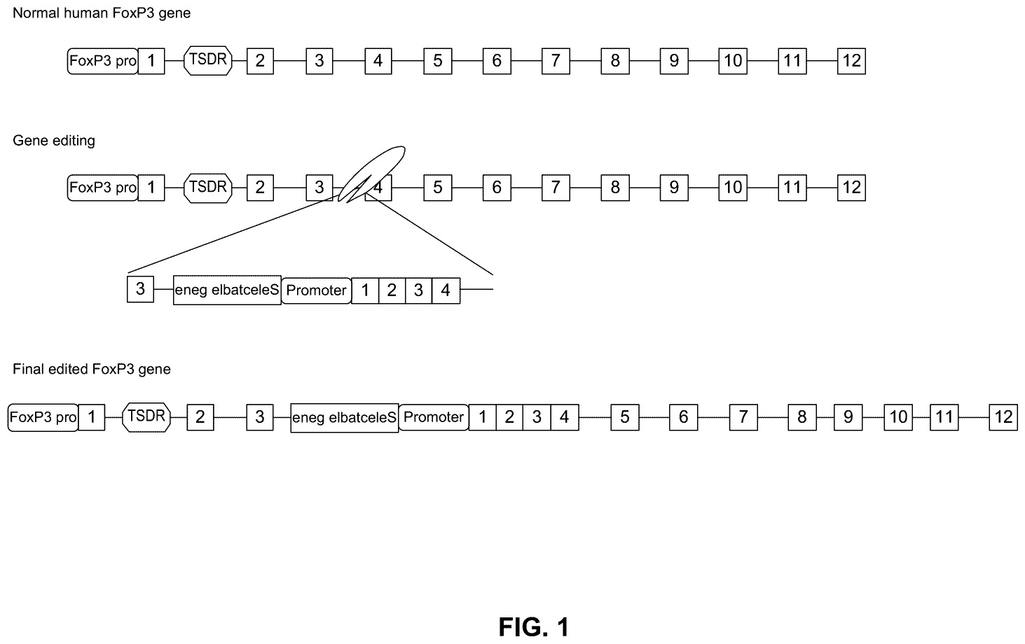

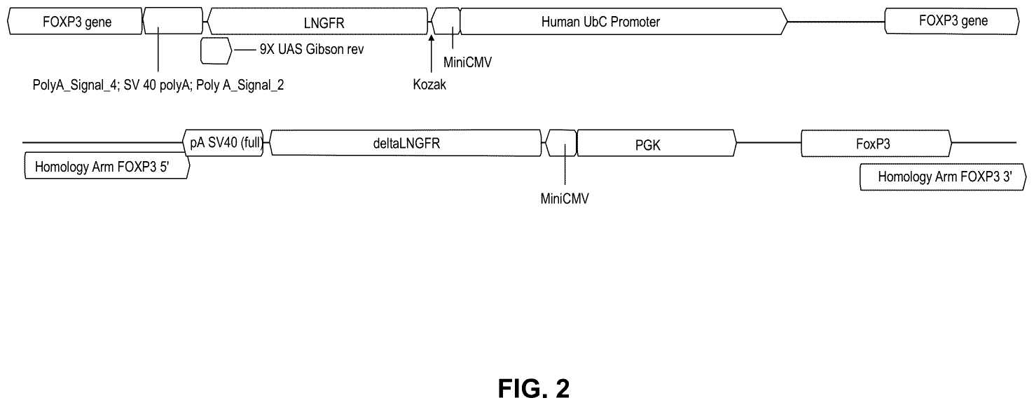

### DETAILED DESCRIPTION

Provided are methods, polynucleotides, and compositions for creating regulatory T cells by gene editing to engineer and reprogram normal T cells into a regulatory phenotype capable of persisting within the body at an increased number. The methods and compositions provided improve upon other methods by increasing the number of regulatory T cells without the need for large scale cellular amplification, which amplification can lead to T cell exhaustion. The methods, polynucleotides, and compositions described herein can be used to treat auto-immunity and prevent and/or treat rejection associated with allo- and xeno-transplantation. In some aspects, Treg cells are generated using targeted, high-efficiency gene-editing to introduce a strong promoter at an endogenous FOXP3 locus by HDR to stably express high quantities of FOXP3 in target cells. In some aspects, the target cells are CD4+ T cells. In some aspects, the target cells are NK T cells. In some aspects, the target cells are hematopoietic stem and progenitor cells. Advantageously, the resulting gene-edited Treg cells exhibit a phenotype and cytokine profile that closely mirrors purified peripheral blood Treg, and manifest immunosuppression in vitro and in vivo.

In some aspects, the methods, polynucleotides and compositions comprise the use of targeted nucleases (for example CRISPR/Cas, Tal-like Effector Nucleases (TALENs), Zinc Finger Nucleases (ZFNs)) to induce a double-strand DNA break within an intron of the FoxP3 gene downstream of the Treg-specific demethylation region (TSDR) region. In some aspects, the double strand DNA break is induced downstream of the 2nd exon within FoxP3. A “donor DNA” template utilizing the cellular repair machinery homes to the site of the break using DNA sequences homologous to the sequences flanking the double strand break site thereby inserting a sequence of synthetic DNA into that site.

### Definitions

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “approximately” or “about” as applied to one or more values of interest, refers to a value that is similar to a stated reference value and within a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about” is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

The terms “ug” and “uM“are used herein interchangeably with “μg” and “μM,” respectively.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “nucleic acids,” “nucleic acid molecules,” “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”, including mRNA) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, DNA plasmid, synthetic DNA, and semi-synthetic DNA. A “nucleic acid composition” of the disclosure comprises one or more nucleic acids as described herein. RNA can be obtained by transcription of a DNA-sequence, e.g., inside a cell. In eukaryotic cells, transcription is typically performed inside the nucleus or the mitochondria. In vivo, transcription of DNA usually results in premature RNA, which has to be processed into messenger RNA (mRNA). Processing of the premature RNA, e.g., in eukaryotic organisms, comprises a variety of different posttranscriptional-modifications such as splicing, 5′-capping, polyadenylation, export from the nucleus or the mitochondria and the like. The sum of these processes is also called maturation of RNA. The mature mRNA usually provides the nucleotide sequence that can be translated into an amino acid sequence of a particular peptide, protein, or protein antigen. Typically a mature mRNA comprises a 5′ cap, optionally a 5′ UTR, an open reading frame, optionally a 3′ UTR, and a poly(A) sequence.

The term “mRNA,” as used herein, refers to a single stranded RNA that encodes the amino acid sequence of one or more polypeptide chains.

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., an shRNA, that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., an shRNA, that includes a region that is substantially complementary to a region of the antisense strand as that term is defined herein. The antisense and sense strands of a dsRNA, e.g., an shRNA, are hybridized to form a duplex structure.

The term “5′” or “5 prime” as used herein refers to the 5′ end of a nucleic acid or nucleic acid sequence, and the term “3′” or “3 prime” as used herein refers to the 3′ end of nucleic acid or nucleic acid sequence.

The term “multicistronic mRNA” or “multicistronic mRNA vector,” as used herein, refers to an mRNA having two or more open reading frames. An open reading frame in this context is a sequence of codons that is translatable into a polypeptide or protein.

The term “5′-cap,” as used herein, refers to an entity, typically a modified nucleotide entity, which generally “caps” the 5′-end of a mature mRNA. A 5′-cap can typically be formed by a modified nucleotide, particularly by a derivative of a guanine nucleotide. In some aspects, the 5′-cap is linked to the 5′-terminus via a 5′-5′-triphosphate linkage. A 5′-cap can be methylated, e.g., m7GpppN, wherein N is the terminal 5′ nucleotide of the nucleic acid carrying the 5′-cap, typically the 5′-end of an RNA. The naturally occurring 5′-cap is m7GpppN.

The term a “poly(A) sequence,” also called “poly(A) tail” or “3′-poly(A) tail,” as used herein is typically understood to be a sequence of adenine nucleotides, e.g., of up to about 400 adenine nucleotides. A poly(A) sequence can be located at the 3′ end of an mRNA. In some aspects, a poly(A) sequence can also be located within an mRNA or any other nucleic acid molecule, such as, e.g., in a vector, for example, in a vector serving as template for the generation of an RNA, preferably an mRNA, e.g., by transcription of the vector. In some aspects, a poly(A) sequence is present in the 3′ UTR of the mRNA as defined herein.

The term “3′ untranslated region” (3′ UTR) as used herein refers to a 3′ UTR sequence that is part of an mRNA, which is located between the protein coding region (i.e., the open reading frame) and the 3′ terminus of the mRNA molecule. If a 3′-terminal poly(A) sequence (‘poly(A) tail’) was added to the RNA (e.g. by polyadenylation), then the term 3′ UTR can refer to that part of the molecule, which is located between the protein coding region and the 3′-terminal poly(A) sequence. In some aspects, a 3′ UTR can also comprise a poly(A) sequence (e.g., a poly(A) sequence which is not located at the very 3′ terminus of the RNA molecule). A 3′ UTR of the mRNA is not translated into an amino acid sequence. The 3′ UTR sequence is generally encoded by the gene, which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′ capping, splicing the pre-mature mRNA to excise optional introns and modifications of the 3′-end, such as polyadenylation of the 3′-end of the pre-mature mRNA and optional endo- or exonuclease cleavages etc. In some aspects, a 3′ UTR corresponds to the sequence of a mature mRNA, which is located 3′ to the stop codon of the protein coding region (e.g., immediately 3′ to the stop codon of the protein coding region), and which extends to the 3′ terminus of the RNA molecule or to the 5′-side of a 3′ terminal poly(A) sequence (e.g., to the nucleotide immediately 5′ to the 3′ terminus or immediately 5′ to the 3′ terminal poly(A) sequence). The term “corresponds to” as used herein means that the 3′ UTR sequence can be an RNA sequence, such as in the mRNA sequence used for defining the 3′ UTR sequence, or a DNA sequence, which corresponds to such RNA sequence. In some aspects, the term “a 3′ UTR of a gene”, is the sequence, which corresponds to the 3′ UTR of the mature mRNA derived from this gene, i.e., the mRNA obtained by transcription of the gene and maturation of the pre-mature mRNA. The term “3′ UTR of a gene” encompasses the DNA sequence and the RNA sequence of the 3′ UTR. In some aspects, the 3′ UTR is derived from a gene that relates to an mRNA with an enhanced half-like (i.e., that provides a stable mRNA), for example a 3′ UTR of a gene selected from the group consisting of: albumin gene, an α-globin gene, a β-globin gene, a tyrosine hydroxylase gene, a lipoxygenase gene, and a collagen alpha gene, such as a collagen alpha 1(I) gene.

A 5′ UTR is typically understood to be a particular section of messenger RNA (mRNA). It is located 5′ of the open reading frame of the mRNA. In some aspect, the 5′ UTR starts with the transcriptional start site and ends one nucleotide before the start codon of the open reading frame. The 5′ UTR can comprise elements for controlling gene expression, also called regulatory elements. Such regulatory elements can be, for example, ribosomal binding sites or a 5′-Terminal Oligopyrimidine Tract. The 5′ UTR can be posttranscriptionally modified, for example by addition of a 5′-cap. In some aspects, a 5′ UTR corresponds to the sequence of a mature mRNA which is located between the 5′ cap and the start codon. In some aspects, the 5′ UTR corresponds to the sequence which extends from a nucleotide located 3′ to the 5′-cap (e.g., from the nucleotide located immediately 3′ to the 5′ cap) to a nucleotide located 5′ to the start codon of the protein coding region (e.g., to the nucleotide located immediately 5′ to the start codon of the protein coding region). The nucleotide located immediately 3′ to the 5′ cap of a mature mRNA typically corresponds to the transcriptional start site. The term “corresponds to” means that the 5′ UTR sequence can be an RNA sequence, such as in the mRNA sequence used for defining the 5′ UTR sequence, or a DNA sequence which corresponds to such RNA sequence. In some aspects, the term “a 5′ UTR of a gene”, is the sequence, which corresponds to the 5′ UTR of the mature mRNA derived from this gene.

As used herein, the terms “derived from” or “derivative” refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of both. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

As used herein, the term “transfecting” or “transfection” refers to the transport of nucleic acids from the environment external to a cell to the internal cellular environment, with particular reference to the cytoplasm and/or cell nucleus. Without being bound by any particular theory, it is to be understood that nucleic acids can be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids can be of human, animal, vegetable, bacterial, viral, or synthetic origin. They can be obtained by any technique known to a person skilled in the art.

As used herein the term “knock in process” refers to a process of introducing one polynucleotide into another polynucleotide (original polynucleotide) such that the resulting polynucleotide contains the introduced polynucleotide and all or part of the original polynucleotide. Nucleases and methodologies for knocking in genes are known in the art and can be used in conjunction with the methods and systems of the invention.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference. A level of a protein can be expressed in mass/vol (e.g., g/dL, mg/mL, g/mL, ng/mL) or percentage relative to total protein or mRNA in a sample.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., an untreated cell or a cell that has not been modified according to the methods described herein, a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated; a sample from a subject that has not been treated by a method as described herein; or a sample of a purified protein (e.g., a selectable marker protein as described herein) at a known normal concentration.

The term “recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed cells, as described further below. The cell expresses the foreign gene to produce the protein under expression conditions.

The terms “recombinant cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

The term “host cell,” as used herein, refers to a cell that can be genetically engineered using the FOXP3 donor constructs described herein and includes T cells, NK T cells, and hematopoietic stem and progenitor cells (HSPCs).

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of mRNA from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “promoter” refers to DNA sequence capable of controlling the expression of a coding sequence or functional RNA. In some aspects, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “bidirectional promoter” refers to a constitutive, regulatable, tissue specific, or ubiquitous promoter that promotes transcription in two opposing directions.

The term “regulatable promoter” as used herein refers to any promoter whose activity is affected by a cis or trans-acting factor (e.g., an inducible promoter, such as an external signal or agent).

The term “constitutive promoter” as used herein refers to any promoter that directs RNA production in many or all tissue/cell types at most times, e.g., the human CMV immediate early enhancer/promoter region that promotes constitutive expression of cloned DNA inserts in mammalian cells.

The term “enhancer” as used herein refers to a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The terms “transcriptional regulatory protein,” “transcriptional regulatory factor,” and “transcription factor” are used interchangeably herein and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to or is bound to a DNA response element.

The term “termination signal sequence” as used herein refers to any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The term “internal ribosome entry site” or “IRES” as used herein refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). “Under translational control of an IRES” as used herein means that translation is associated with the IRES and proceeds in a cap-independent manner.

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. The term “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

The term “additional proteolytic cleavage site” as used herein refers to a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary “additional proteolytic cleavage sites” are described herein and include, but are not limited to, furin cleavage sites with the consensus sequence RXK(R)R (SEQ ID NO: 17). Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “coding sequence” or a sequence “encoding” a particular molecule (e.g., a protein, e.g., a FOXP3 protein or a selectable marker protein) as used herein refer to a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “vector,” as used herein, is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. In some aspects, such vectors include, but are not limited to: an adenoviral vector, an adeno-associated virus (AAV) vector, retroviral vector, a lentiviral vector, poxvirus vector, a baculovirus vector, a herpes viral vector, simian virus 40 (SV40), cytomegalovirus (CMV), mouse mammary tumor virus (MMTV), and Moloney murine leukemia virus. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can sometimes be used interchangeably, depending on the context, as the plasmid is the most commonly used form of vector. However, also disclosed herein are other forms of expression vectors, such as viral vectors (e.g., replication defective retroviruses, poxviruses, herpesviruses, baculoviruses, adenoviruses and adeno-associated viruses), which can serve equivalent functions.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably. In some aspects, the AAV vector is modified relative to the wild-type AAV serotype sequence.

As used herein, an “AAV particle” is an AAV virus that comprises an AAV vector having at least one payload region (e.g., a polynucleotide encoding a therapeutic protein or peptide) and at least one inverted terminal repeat (ITR) region. In some aspects, the terms “AAV vectors of the present disclosure” or “AAV vectors” refer to AAV vectors comprising a polynucleotide encoding a FOXP3 protein and/or a selectable marker protein, e.g., encapsulated in an AAV particle.

As used herein, a “gene therapy composition” is a composition comprising a polynucleotide or a vector comprising a polynucleotide, wherein the polynucleotide or vector comprises, e.g., a FOXP3 donor construct and, optionally, a polynucleotide expressing a nuclease.

The phrase “contacting a cell” (e.g., contacting a cell with an AAV vector, an AAV capsid, or the gene therapy composition of the disclosure) as used herein, includes contacting a cell directly or indirectly. In some aspects, contacting a cell with an AAV vector, an AAV capsid, or the gene therapy composition includes contacting a cell in vitro with the gene therapy composition, the AAV vector, or the AAV capsid or contacting a cell in vivo with the AAV vector, the AAV capsid, or the gene therapy composition. Thus, for example, the AAV vector, the AAV capsid, or the gene therapy composition can be put into physical contact with the cell by the individual performing the method, or alternatively, the AAV vector, the AAV capsid, or the gene therapy composition can be put into a situation that will permit or cause it to subsequently come into contact with the cell.

In some aspects, contacting a cell in vitro can be done, for example, by incubating the cell with the AAV vector. In some aspects, contacting a cell in vivo can be done, for example, by injecting the AAV vector, the AAV capsid, or the gene therapy composition of the disclosure into or near the tissue where the cell is located (e.g., bone marrow, thymus), or by injecting the AAV vector, the AAV capsid, or the gene therapy composition into another area, e.g., the bloodstream or the subcutaneous space, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with an AAV vector, an AAV capsid, or the gene therapy composition and subsequently transplanted into a subject.

In some aspects, contacting a cell with a polynucleotide, expression cassette, vector, rAAV particle, nuclease protein or composition of the disclosure includes “introducing” or “delivering” (directly or indirectly) the AAV vector, the AAV capsid, the nuclease protein or the gene therapy composition into the cell by facilitating or effecting uptake or absorption into the cell. Introducing an AAV vector, an AAV capsid, a nuclease protein or the gene therapy composition into a cell can be in vitro and/or in vivo. For example, for in vivo introduction, an AAV vector, an AAV capsid, a nuclease protein or the gene therapy composition can be injected into a specific tissue site (e.g., the locus where a therapeutic effect is desired) or administered systemically (e.g., administering a AAV vector targeted to a locus where a therapeutic effect is desired). In vitro introduction into a cell includes methods known in the art such as electroporation and lipofection.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal or cell or tissue thereof).

The term “mutation” as used herein refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

The term “modified” as used herein refers to a changed state or structure of a molecule of the disclosure. Molecules can be modified in many ways including chemically, structurally, and functionally. In some aspects, the modification is relative to a reference wild-type molecule.

The term “synthetic” as used herein refers to produced, prepared, and/or manufactured by the hand of man. Synthesis of polynucleotides or polypeptides or other molecules of the present disclosure can be chemical or enzymatic.

The term “polypeptide” as used herein is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an albumin domain, to increase half-life. A peptide as described herein can also be derivatized in a different ways. A peptide described herein can comprise modifications including e.g., conjugation of a palmitoyl group.

The term “homology-directed repair” (HDR) as used herein refers to a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels, PNAS (0027-8424), 111 (10), p. E924-E932).

The term “nuclease” as used herein refers to an enzyme that possesses catalytic activity for DNA cleavage. In some aspects, a nuclease can promote homologous recombination between a FOXP3 construct disclosed herein and a gene, e.g., a FOXP3 gene. In some aspects, the construct to be integrated in the genome of a host cell contains regions of homology adjacent to a sequence targeted by a nuclease, e.g., a CRISPR/Cas nuclease.

The term “administration” as used herein refers to the administration of a composition of the present disclosure (e.g., an AAV vector, an AAV capsid, or the gene therapy composition disclosed herein) to a subject or system. Administration to a subject (e.g., to a human) can be by any appropriate route, such as but not limited to periorbital, retrobulbar and/or intramuscular injection.

The term “inverted terminal repeat” (or “ITR”) as used herein refers to a single stranded sequence of nucleotides followed downstream by its reverse complement. The intervening sequence of nucleotides between the initial sequence and the reverse complement can be any length including zero.

The term “tolerogenic” as used herein means capable of suppressing or down-modulating an adaptive or innate immunological response.

The term “biological sample” as used herein refers any sample obtained from an organism and encompasses a clinical sample. The types of “biological samples” include, but are not limited to: tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like. In some aspects, the biological sample comprises hematopoietic cells. In some aspects, the biological sample comprises hematopoietic progenitor or stem cells. In some aspects, the biological sample comprises T cells or NK T cells.

The terms “obtained” or “obtaining” as used herein refer to a physical extraction or isolation of a biological sample (e.g., comprising hematopoietic stem and progenitor cells (HSPCs), lymphoid progenitors, peripheral blood mononuclear cells (PBMCs), CD4+T lymphocytes, or NK T cells) from a subject. For example, a biological sample comprises hematopoietic cells isolated from a subject (and thus “obtained”) by the same person or same entity that subsequently isolates HSPC, CD4+T lymphocytes, NK T cells etc. from the sample and produces FOXP3 engineered T cells (gene edited with CRISPR/Cas9 and FOXP3 homology donor vectors) from the original unmodified cells in the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample. In some embodiments, the step of obtaining comprises the step of isolating a biological sample (e.g., a pre-treatment biological sample, a post-treatment biological sample, etc.). Methods and protocols for isolating various biological samples (e.g., a blood sample, a biopsy sample, an aspirate, etc.) will be known to one of ordinary skill in the art and any convenient method may be used to isolate a biological sample.

The phrase “substantially purified” generally refers to isolation of a component of a sample (e.g., cell or substance), such that the component comprises the majority percent of the sample in which it resides. Typically, in a sample, a substantially purified component comprises at least 70%, preferably at least 80%-85%, more preferably at least 90-99% of the sample.

The terms “treatment”, “treating”, “treat” and the like as used herein refer generally to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or one or more symptom(s) from occurring in a subject who may be predisposed to the disease or symptom but has not yet been diagnosed as having it; (b) inhibiting the disease and/or one or more symptom(s), i.e., arresting their development; or (c) relieving one or more of the disease symptom(s), i.e., causing regression of the disease and/or one or more symptom(s). Those in need of treatment include those already inflicted as well as those in which prevention is desired (e.g., those with increased susceptibility to an autoimmune disease, etc.) In some aspects, preventing an outcome is achieved through prophylactic treatment. As used herein, “prophylactic” refers to a therapeutic or course of action used to prevent the onset of a disease or condition, or to prevent or delay one or more symptoms associated with a disease or condition. As used herein, a “prophylaxis” refers to a measure taken to maintain health and prevent or delay the onset of a disease or condition, or to prevent or delay one or more symptoms associated with a disease or condition.

A therapeutic treatment is one in which the subject is inflicted prior to administration and a prophylactic treatment is one in which the subject is not afflicted prior to administration. In some aspects, the subject has an increased likelihood of becoming inflicted or is suspected of being inflicted prior to treatment. In some embodiments, the subject is suspected of having an increased likelihood of becoming inflicted.

The term “pharmaceutically acceptable excipient or carrier” as used herein refers to an excipient that may optionally be included in the compositions of the disclosure and that causes no significant adverse toxicological effects to the patient.

The term “pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term an “effective amount” of a composition comprising FOXP3 engineered HSPC or FOXP3 engineered T cells or NK T cells (e.g., cells edited with CRISPR/Cas9 and FOXP3 homology donor vectors) as used herein refers to an amount sufficient to safely effect beneficial or desired results, such as an amount that suppresses activation and proliferation of effector T cells and increases immune tolerance. An effective amount can be administered in one or more administrations, applications, or dosages.

The term “therapeutically effective dose or amount” of a composition comprising FOXP3 engineered HSPC or FOXP3 engineered T cells or NK T cells as used herein refers to an amount that, when administered as described herein, brings about a positive therapeutic response, such as improved recovery from an inflammatory condition such as, but not limited to, an autoimmune manifestation, an allergy, graft-versus-host disease, and transplant rejection. Improved recovery may include a reduction in inflammation, pain, or autoimmune-induced tissue damage, or better graft tolerance and prolonged survival of transplanted cells, tissue or organs. Additionally, a therapeutically effective dose or amount may compensate for functional (e.g., IPEX syndrome) or quantitative Treg-deficiency and reduce the need for immunosuppressive or anti-inflammatory drugs. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein. For example, an effective unit dose may be about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁷ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, about 9×10⁷ cells/kg, about 1×10⁸ cells/kg, about 2×10⁸ cells/kg, about 3×10⁸ cells/kg, about 4×10⁸ cells/kg, about 5×10⁸ cells/kg, about 6×10⁸ cells/kg, about 7×10⁸ cells/kg, about 8×10⁸ cells/kg, about 9×10⁸ cells/kg, about 1×10⁹ cells/kg, about 2×10⁹ cells/kg, about 3×10⁹ cells/kg, about 4×10⁹ cells/kg, about 5×10⁹ cells/kg, about 6×10⁹ cells/kg, about 7×10⁹ cells/kg, about 8×10⁹ cells/kg, about 9×10⁹ cells/kg, about 1×10¹⁰ cells/kg or a range bounded by any of these numbers; or from about 1×10⁵ cells/kg to about 1×10¹⁰ cells/kg, or about 1×10⁵ cells/kg to about 1×10⁶ cells/kg, about 2×10⁶ cells/kg to about 1×10⁷ cells/kg, about 2×10⁷ cells/kg to about 1×10⁸ cells/kg, about 2×10⁸ cells/kg to about 1×10⁹ cells/kg, about 2×10⁹ cells/kg to about 1×10¹⁰ cells/kg, or more.

The terms “recipient,” “individual,” “subject,” “host, and “patient” as used herein are interchangeable and refer to any mammalian subject for whom diagnosis, treatment, or therapy is desired, such as humans, domestic or farm animals, and zoo, sports, or pet animals, such as dogs, horses, cats, cows, sheep, goats, pigs, etc.

The term “unit dosage form,” as used herein, refers to physically discrete units suitable as unitary dosages for human and non-human subjects, each unit containing a predetermined quantity of the agents calculated in an amount sufficient to produce the desired effect in association with a pharmaceutically acceptable diluent, carrier or vehicle. The specifications for the unit dosage forms for use in the present disclosure depend on the particular compound employed and the effect to be achieved, the pharmacodynamics associated with each compound in the host, and the like.

### Nucleases

In some aspects, the FOXP3 donor construct to be integrated in the genome of a host cell (e.g., a T cell) contains regions of homology adjacent to a sequence (e.g., a recognition site) targeted by a nuclease, e.g., a CRISPR/Cas nuclease.

The size of the recognition site for the nuclease mediating homologous recombination can vary depending on the nuclease, and includes, for example, recognition sites that are at least about 4, at least about 6, at least about 8, at least about 10, at least about 12, at least about 14, at least about 16, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 nucleotides in length; or about 4 nucleotides to about 50 nucleotides; about 4 nucleotides to about 40 nucleotides, about 4 nucleotides to about 36 nucleotides, about 4 nucleotides to about 32 nucleotides, about 4 nucleotides to about 28 nucleotides, about 4 nucleotides to about 24 nucleotides, about 4 nucleotides to about 20 nucleotides, about 4 nucleotides to about 16 nucleotides, about 4 nucleotides to about 12 nucleotides, about 4 nucleotides to about 10 nucleotides, about 4 nucleotides to about 8 nucleotides, about 4 nucleotides to about 6 nucleotides; or about 6 nucleotides to about 40 nucleotides, about 6 nucleotides to about 36 nucleotides, about 6 nucleotides to about 32 nucleotides, about 6 nucleotides to about 28 nucleotides, about 6 nucleotides to about 24 nucleotides, about 6 nucleotides to about 20 nucleotides, about 6 nucleotides to about 16 nucleotides, about 6 nucleotides to about 12 nucleotides, about 6 nucleotides to about 10 nucleotides or about 6 nucleotides to about 8 nucleotides.

In one aspect, each monomer of the nuclease recognizes a recognition site of at least 9 nucleotides. In other aspects, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides). The recognition site could be palindromic, that is, the sequence on one strand reads the same in the opposite direction on the complementary strand. It is recognized that a given nuclease can bind the recognition site and cleave at or near the binding site. The cleavage by the nuclease can occur at nucleotide positions immediately opposite each other to produce a blunt end cut or, in other cases, the cleavage sites can be staggered to produce single-stranded overhangs, also called “sticky ends,” which can be either 5′ overhangs, or 3′ overhangs.

Any nuclease that induces a nick or double-strand break into a desired recognition site can be used in the methods and compositions disclosed herein. A naturally-occurring nuclease can be employed so long as the nuclease induces a nick or double-strand break at or near a desired recognition site. Alternatively, a modified or engineered nuclease can be employed. An “engineered nuclease” comprises a nuclease that is derived from its natural form and has been modified to specifically recognize and induce a nick or double-strand break in a desired recognition site. The modification of the nuclease can be as little as one amino acid in a nuclease protein or one nucleotide in a nucleic acid encoding the nuclease. In some aspects, the engineered nuclease induces a nick or double-strand break in a recognition site, wherein the recognition site was not a sequence that would have been recognized by a non-engineered nuclease. Producing a single-strand break or double-strand break in a DNA can be referred to herein as “nicking” or “cleaving” the DNA, respectively.

In some aspects of the present disclosure, the homologous recombination is initiated by a CRISPR/Cas system, a TALEN system, a ZFN system, a meganuclease, or a restriction endonuclease.

In some aspects, the nuclease is introduced into the cell by any means known in the art. For example, for CRISPR/Cas systems a ribonucleoprotein (RNP) comprising a Cas9 protein in complex with a trans-activating CRISPR RNA and CRISPR RNA (tracrRNA:crRNA-Cas9) can be introduced directly into a cell. Alternatively, a polynucleotide(s) encoding Cas9 and the tracrRNA and crRNA can be introduced into a cell. If expressed from a polynucleotide, the CRISPR RNAs and the Cas9 can be expressed from different promoters.

Active variants and fragments of nuclease (i.e., an engineered nuclease) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease, wherein the active variants retain the ability to cut at a desired recognition site and hence retain nick or double-strand-break-inducing activity. For example, any of the nuclease described herein can be modified from a native endonuclease sequence and designed to recognize and induce a nick or double-strand break at a recognition site that was not recognized by the native nuclease. Thus in some aspects, the engineered nuclease has a specificity to induce a nick or double-strand break at a recognition site that is different from the corresponding native nuclease recognition site. Assays for nick or double-strand-break-inducing activity are known and generally measure the overall activity and specificity of the endonuclease on DNA substrates containing the recognition site.

When the nuclease is provided to the cell through the introduction of a polynucleotide encoding the nuclease, such a polynucleotide can be codon-optimized for expression in that cell. In aspects, the polynucleotide encoding a nuclease can be modified to substitute codons having a higher frequency of usage in the cell of interest, as compared to the naturally occurring polynucleotide sequence encoding the nuclease. For example, the polynucleotide encoding the nuclease can be modified to substitute codons having a higher frequency of usage in a given prokaryotic or eukaryotic cell of interest, including a bacterial cell, a yeast cell, a human cell, or any other host cell of interest, as compared to the naturally occurring polynucleotide sequence.

### CRISPR/Cas

In some aspects, the nuclease employed in the various methods and compositions disclosed herein can comprise a CRISPR/Cas system. Such systems can employ, for example, a Cas9 nuclease, which in some instances, is codon-optimized for the desired cell type in which it is to be expressed. Such systems can also employ a guide RNA (gRNA) that comprises two separate molecules. An exemplary two-molecule gRNA comprises a crRNA-like (“Clustered Regularly Interspaced Short Palindromic Repeats (CRISPR) RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence, and a tracrRNA. The target sequence is upstream of a protospacer adjacent motif (PAM) sequence. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used. The gRNA is also referred to herein as a CRISPR guide sequence.

Alternatively, the system further employs a fused crRNA-tracrRNA construct (i.e., a single transcript, optionally including intervening nucleotides linking the crRNA and tracrRNA) that functions with the Cas9. This single RNA is often referred to as a single-guide RNA or sgRNA. Within a sgRNA, the crRNA portion is identified as the “targeter sequence” for the given recognition site and the tracrRNA is often referred to as the “scaffold.” Briefly, a short DNA fragment containing the targeter sequence can be inserted into a guide RNA expression plasmid. The gRNA expression plasmid may comprise the targeter sequence (in some aspects around 20 nucleotides), a form of the tracrRNA sequence (the scaffold) as well as a suitable promoter that is active in the cell

The gRNA expression cassette and the Cas9 expression cassette are then introduced into the cell. See, for example, Mali P et al. (2013) Science 2013 Feb. 15; 339(6121):823-6; Jinek M et al. Science 2012, published online Jun. 28, 2012; 337(6096):816-21; Hwang W Y et al. Nat Biotechnol 2013 March; 31(3):227-9; Jiang W et al. Nat Biotechnol 2013 March; 31(3):233-9; and Cong L et al. Science 2013 Feb. 15; 339(6121):819-23, each of which is herein incorporated by reference. See also the various commercially available kits and, for example, WO/2013/176772A1, WO/2014/065596A1, WO/2014/089290A1, WO/2014/093622A2, WO/2014/099750A2, and WO/2013142578A1, each of which is herein incorporated by reference.

In some aspects, an alternative Cas protein can be used. For example, a Cas12, or Cas13 protein can be used. In some aspects, the Cas9 nuclease can be provided in the form of a protein. In some aspects, the Cas9 protein can be provided in the form of a ribonucleoprotein (RNP) complex with the gRNA. In other aspects, the Cas9 nuclease can be provided in the form of a nucleic acid encoding the protein. The nucleic acid encoding the Cas9 nuclease can be RNA (e.g., messenger RNA (mRNA)) or DNA. In some aspects, the gRNA can be provided to the cell in the form of RNA. In other aspects, the gRNA can be provided to the cell in the form of DNA encoding the RNA. In some aspects, the gRNA can be provided in the form of separate crRNA and tracrRNA molecules, or separate DNA molecules encoding the crRNA and tracrRNA, respectively. In some aspects, the gRNA is referred to as a CRISPR guide sequence and is provided in the form of RNA. In some aspects, the CRISPR guide sequence is provided in the form of DNA encoding the RNA. In some aspects, the CRISPR guide sequence can be provided in the form of separate crRNA and tracrRNA molecules.

In some aspects, the Cas protein is a type I Cas protein (see, e.g., Xu et al., Environmental Microbiology 23: 542-558, 2021). In one aspect, the Cas protein is a type II Cas protein. In one aspect, the type II Cas protein is Cas9. In one aspect, the type II Cas, e.g., Cas9, is a human codon-optimized Cas9.

In certain aspects, the Cas protein is a “nickase” that can create single strand breaks (i.e., “nicks”) at the target site without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, in WO/2013/176772A1 and WO/2013/142578A1, each of which is herein incorporated by reference.

In certain aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

### TALENs

In some aspects, the nuclease employed in the various methods and compositions disclosed herein can comprise a TALEN. Thus, in one aspect, the nuclease is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism or cell(s). TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

Examples of suitable TAL nucleases, and methods for preparing suitable TALENs, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are designed to bind at or near target nucleic acid sequences to be modified by targeting vectors as described herein.

In one aspect, each monomer of the TALEN comprises 12-25 TAL repeats, wherein each TAL repeat binds a 1 bp subsite. In one aspect, the nuclease is a chimeric protein comprising a TAL repeat-based DNA binding domain operably linked to an independent nuclease. In one aspect, the independent nuclease is a FokI endonuclease. In one aspect, the nuclease comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In one aspect, the nuclease comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

### Zinc-Finger Nucleases (ZFN)

In some aspects, the nuclease employed in the various methods and compositions disclosed herein can comprise a zinc-finger nuclease (ZFN) system. In one aspect, each monomer of the ZFN comprises 3 or more zinc finger-based DNA binding domains, wherein each zinc finger-based DNA binding domain binds to a 3 bp subsite. In other aspects, the ZFN is a chimeric protein comprising a zinc finger-based DNA binding domain operably linked to an independent nuclease. In one aspect, the independent endonuclease is a FokI endonuclease. In one aspect, the nuclease comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, for example, US20060246567; US20080182332; US20020081614; US20030021776; WO/2002/057308A2; US20130123484; US20100291048; and, WO/2011/017293A2, each of which is herein incorporated by reference.

### Meganucleases

In some aspects, the nuclease employed in the various methods and compositions disclosed herein can comprise a meganuclease system. Meganucleases have been classified into four families based on conserved sequence motifs, the families are the “LAGLIDADG,” “GIY-YIG,” “H—N—H,” and “His-Cys box” families. These motifs participate in the coordination of metal ions and hydrolysis of phosphodiester bonds.

In some aspects, the meganucleases are Homing Endonucleases (HEases) notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some aspects, naturally occurring variants, and/or engineered derivative meganucleases are used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcITP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In some aspects, the meganuclease recognizes double-stranded DNA sequences of 12 to 40 base pairs. In some aspects, the meganuclease recognizes one perfectly matched target sequence in one of the heterologous polynucleotides described herein. In some aspects, the meganuclease is a homing nuclease. In some aspects, the homing nuclease is a member of the “LAGLIDADG” family of homing nuclease. In some aspects, homing nucleases is selected from I-SceI, I-CreI, and I-Dmol.

### Restriction Endonucleases

In some aspects, the nuclease employed for homologous recombination in the various methods and compositions disclosed herein can comprise a restriction endonuclease, which includes Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

### Polynucleotides

In some aspects, polynucleotides comprising a gene editing system for editing a FOXP3 gene are provided. The gene editing system comprises a nuclease such as a zinc-finger nuclease (ZFN), a transcription activator-like effector nuclease (TALEN), CRISPR/Cas system, a dimeric CRISPR RNA guided Fok1 nuclease, a meganuclease or a restriction endonuclease, and the nuclease is introduced into a cell, e.g., a T cell, a NK T cell or a HSPC together with a FOXP3 donor polynucleotide construct.

In some aspects, the polynucleotide encodes a TAL effector nuclease that is engineered to bind to and cut in or near a target nucleic acid sequence in, e.g., a FOXP3 intron or exon.

In some aspects, the polynucleotide encodes a zinc-finger nuclease or a transcription activator-like effector nuclease (TALEN). The zinc-finger nuclease or TALEN bind to specific sequences upstream and/or downstream of an intron or exon of the FOXP3 gene via a DNA binding polypeptide (see, e.g., Carlson et al. (2012), Targeting DNA with fingers and TALENs, Molecular Therapy-Nucleic Acids 1, e3).

In some aspects, the polynucleotide encodes a Cas9 nuclease and guide RNA, which bind to an intron or exon of a FOXP3 gene (see, e.g., Mail et al. (2013), RNA-guided human genome engineering via Cas9, Science 339(6121), pages 823-826; Cong et al. (2013), Multiplex genome engineering using CRISPR/Cas systems, Science 339(6121), pages 819-823; and Tsai et al. (2014), Dimeric CRISPR RNA-guided FokI nucleases for highly specific genome editing, Nature Biotechnology 32, p. 569-576.

In some aspects, the crRNA target sequence is: ATCCACCGTTGAGAGCTGGG (SEQ ID NO: 1). This target sequence is located within the FOXP3 gene and spans the exon 4 splice site with the target cut site within intron 3 (nucleotides 7160-1741 reverse orientation), which reduces the possibility of deleterious mutations arising from a cut at the target sequence of SEQ ID NO: 1 without subsequent donor construct insertion. The region that comprises the target sequence of SEQ ID NO: 1 is removed from the natural regulatory mechanisms for inhibiting FOXP3 expression (mechanisms that rely, e.g., on modifications to the DNA nucleotides themselves). Furthermore, the target sequence of SEQ ID NO: 1 is a unique sequence without closely related sequences within the human genome reducing the risk of adverse effects at other sites in the human genome. Following this strategy, additional crRNA target sequences within the FOXP3 gene can be generated and used in the methods described herein.

In some aspects, a polynucleotide donor construct comprises a polynucleotide comprising a nucleic acid of a portion of a FOXP3 intron and/or a portion of a FOXP3 exon or, alternatively, the entire FOXP3 gene, and a promoter. In some aspects, the polynucleotide further comprises a polynucleotide encoding a selectable marker. In some aspects, the polynucleotide comprising a nucleic acid of a portion of a FOXP3 intron and/or a portion of a FOXP3 exon or the entire FOXP3 gene and the polynucleotide encoding the selectable marker are located on opposite ends of a bidirectional promoter and are under the control of said bidirectional promoter.

In some aspects, a polynucleotide donor construct comprises a polynucleotide comprising a nucleic acid of a portion of a FOXP3 exon 3, a portion of a FOXP3 intron 3, a nucleic acid encoding a marker gene, a promoter, and a nucleic acid of at least exons 1 and 2 of a FOXP3 gene, and a portion of a FOXP3 intron 4. In some aspects, the promoter of the donor construct is bidirectional and the marker gene is oriented such that it is operably linked to the bidirectional promoter. In some aspects, the marker gene and the first FOXP3 exon are operably linked to the bidirectional promoter such that their transcription proceeds in opposite directions.

In some aspects, a polynucleotide donor construct comprises a polynucleotide comprising a nucleic acid of a portion of a FOXP3 exon 3, a portion of a FOXP3 intron 3, a nucleic acid encoding a marker gene, a bidirectional promoter, a nucleic acid of an exon 1, an exon 2, and an exon 3 of a FOXP3 gene, and a portion of a FOXP3 intron 4.

In some aspects, a polynucleotide donor construct comprises a polynucleotide comprising a nucleic acid of a portion of a FOXP3 exon 3, a portion of a FOXP3 intron 3, a nucleic acid encoding a marker gene, a bidirectional promoter, a nucleic acid of an exon 1, an exon 2, an exon 3 and an exon 4 of a FOXP3 gene, and a portion of a FOXP3 intron 4.

In some aspects, the FOXP3 donor construct is exchanged by homologous recombination (HR) for a section of a genomic FOXP3 intron and/or exon, or portions thereof.

In some aspects, upon homologous recombination at the target sequence of SEQ ID NO: 1, and insertion of the donor construct comprising a nucleic acid of a portion of a FOXP3 exon 3, a portion of a FOXP3 intron 3, a nucleic acid encoding a marker gene, a bidirectional promoter, a nucleic acid of an exon 1, an exon 2, an exon 3, and an exon 4 of a FOXP3 gene, and a portion of a FOXP3 intron 4, a cell genome comprises an engineered FOXP3 gene comprising exons 1-12 under the control of an exogenous promoter. In some aspects, exons 1-12 of the engineered FOXP3 gene are operably linked to the promoter without any intervening coding sequences such as, e.g., marker gene sequences.

In some aspects, the location of the exchange is predetermined by the homology arms of the FOXP3 donor construct. In some aspects, the FOXP3 donor construct comprises a FOXP3 homology arm on its 5′ end and a FOXP3 homology arm on its 3′ end. In some aspects, the homology arms of the FOXP3 construct are homologous to nucleic acid sequences on both sides of the double strand break induced by the nuclease as described herein such that a hybridization and homologous recombination can take place after the nuclease has induced the double strand break. As a consequence of the homologous recombination of the FOXP3 donor construct with the genomic FOXP3 site, the FOXP3 gene is permanently modified to comprise the FOXP3 donor construct.

In some aspects, if a protospacer adjacent motif (PAM) and CRISPR target site are present in a FOXP3 donor construct described herein, such site can be removed by mutating the PAM sequence to prevent Cas9-mediated cleavage of the FOXP3 donor construct. For example, in one donor construct described herein, a PAM sequence present in the FOXP3 portion of the FOXP3 donor construct is disrupted by a silent mutation (G7135A) in the terminal nucleotide of the invariant PAM for Cas9, NGG, to prevent Cas9-mediated cleavage within the respective FOXP3 donor construct.

In some aspects, the FOXP3 donor construct comprises a bidirectional promoter located between a FOXP3 intron and/or exon sequence of the FOXP3 donor construct and a polynucleotide encoding a selectable marker gene.

In some aspects, the selectable marker gene is any gene that can discern a FOXP3 donor construct engineered cell from a non-FOXP3 donor construct engineered cell. For example, the selectable marker gene can be a gene whose encoded protein is not present on a cell that is targeted by the FOXP3 donor construct. In some aspects, the selectable marker gene can be a gene whose encoded protein is present on a cell that is targeted by the FOXP3 donor construct in very low amounts, such as, e.g., in amounts that are not detectable by a cell surface protein labeling assay.

For example, in some aspects, the cell engineered with the FOXP3 donor construct is a T cell, a NK T cell or a HSPC and the selectable marker gene is a neuronal cell-specific protein. In some aspects, the cell engineered with the FOXP3 donor construct is a hematopoietic stem cell and the selectable marker gene is a protein only expressed in a non-hematopoietic stem cell. These examples are for illustration of the selectable marker gene in principle and are not to be understood as limited to the exemplary disclosures. A person of ordinary skill in the art readily can devise selectable marker genes for other engineered cell types based on the knowledge in the art of expression patterns of cell types.

In some aspects, the engineered cell is a T cell and the selectable marker gene encodes a truncated neuronal growth factor receptor protein (e.g., low affinity NGFR, LNFGR).

In some aspects, the FOXP3 donor construct polynucleotide is packaged into an adeno-associated viral vector (AAV), and/or is encoded by a plasmid DNA, and/or is packed into a lentiviral vector, and/or is packed into a protein-capped adenoviral vector (AdV). AAV, lentiviral and AdV vectors have been proven successful in the practice of gene transfer because of the absence of gene toxic side effects.

In some aspects of the disclosure, a CRISPR gene editing system is used to generate a FOXP3 expressing cell. CRISPR based genome editing methods provide advantages over traditional lentiviral methods of gene addition. Advantages include, but are not limited to, targeted introduction of a promoter of choice in the endogenous FOXP3 gene and regulatable or constitutive expression of FOXP3 in gene edited cells.

In some aspects, the FOXP3 homology donor vector comprises a CRISPR/Cas9 polynucleotide comprising a CRISPR/Cas9 system that cuts the endogenous FOXP3 gene at the target site of a sgRNA. After cutting, the FOXP3 homology donor vector then replaces portions of, or the entire, endogenous copy of FOXP3 gene with a desired FOXP3 polynucleotide donor construct contained within the FOXP3 homology polynucleotide donor vector using homology directed repair in a hematopoietic cell, converting the cell into a gene edited cell. For example, nucleic acids encoding portions of, or the entire, FOXP3 transcription factor can be inserted into the FOXP3 homology donor vector to create a vector capable of replacing the endogenous copy of FOXP3 with a FOXP3 gene that contains a selectable marker gene inserted between the Treg-specific demethylation region (TSDR) and the FOXP3 second exon or between the FOXP3 second and third exons or between the FOXP3 third and fourth exons, or between the FOXP3 fourth and fifth exons, or between the FOXP3 fifth and sixth exons, or between the FOXP3 sixth and seventh exons, or between the FOXP3 seventh and eighth exons, or between the FOXP3 eighth and ninth exons, or between the FOXP3 ninth and tenth exons, or between the FOXP3 tenth and eleventh exons, or between the FOXP3 eleventh and twelfth exons.

In some aspects, the recombinant FOXP3 homology donor vector comprises: a) a 5′ homology arm; b) a polynucleotide encoding portions of, or the entire FOXP3 or a variant thereof; c) optionally, a polyadenylation sequence; d) a polynucleotide encoding a selectable marker; e) at least one promoter, wherein the promoter is operably linked to the polynucleotide encoding portions of, or the entire, FOXP3 or a variant thereof and the polynucleotide encoding the cell surface marker; and f) a 3′ homology arm.

In some aspects, the FOXP3 homology donor vector comprises more than one promoter, wherein one promoter is operable linked to the polynucleotide encoding portions of, or the entire, FOXP3 or a variant thereof and one promoter is operably linked to the polynucleotide encoding the selectable marker.

In some aspects, the FOXP3 homology donor vector comprises one promoter, wherein the promoter is bidirectional and promotes transcription of the polynucleotide encoding portions of, or the entire, FOXP3 or a variant thereof and the polynucleotide encoding the selectable marker.

In some aspects, the bidirectional promoter is selected from Elongation Factor 1 alpha (EF1α)-cytomegalovirus (CMV) enhancer/actin promoter, EF1α-phosphoglycerate kinase (PGK) enhance/PGK minimal promoter, EF1α-PGK enhancer/2×PGK minimal promoter, EF1α-PGK enhancer/4×PGK minimal promoter, EF1α-CMV enhancer/2×EF1α promoter, EF1α-PGK enhancer/2×EF1α promoter, EF1α-PGK enhancer/4×EF1α promoter, PGK minimal promoter-PGK enhancer/4 PGK minimal promoter, beta-actin-beta-globin (AG)-CMV enhancer/2× actin promoter, AG-CMV enhancer/4× actin promoter, actin promoter-CMV enhancer/PGK enhancer/2×CMV enhancer/actin promoter, actin promoter-CMV enhancer/PGK enhancer/4×CMV enhancer/actin promoter, actin promoter-CMV enhancer/2×PGK enhancer/2×CMV enhancer/2× actin promoter, or actin promoter-CMV enhancer/3×PGK enhancer/2×CMV enhancer/3× actin promoter.

In some aspects, the bidirectional promoter is a Ubiquitin promoter. In some aspects, the bidirectional promoter is a Ubiquitin C promoter. In some aspects, the bidirectional promoter is a human Ubiquitin C promoter. In some aspects, the Ubiquitin C promoter comprises a minimal CMV promoter element.

In some aspects, the bidirectional promoter is a PGK promoter. In some aspects, the bidirectional promoter is a human PGK promoter. In some aspects, the human PGK promoter comprises a minimal CMV promoter element.

In some aspects, the bidirectional promoter is a constitutive promoter. In some aspects, the bidirectional promoter is a regulatable promoter. In some aspects, the bidirectional promoter is a tissue-specific promoter. In some aspects, the bidirectional promoter is a ubiquitous promoter.

In some aspects, the bidirectional promoter is a regulatable promoter and comprises multiple tet operator sequences (tetO) of the Escherichia coli Tn10 tetracycline resistance operon flanked by two minimal promoters. In some aspects, the minimal promoters each comprise sequence positions −53 to +75 of the human cytomegalovirus IE promoter (hCMV IE promoter). In some aspects, the regulatable promoter comprises 1-10 tet operators. In some aspects, the regulatable promoter comprises 7 tet operators. In some aspects, the cell comprising the regulatable promoter further comprises a fusion between a Tet repressor (TetR) and a herpes simplex virus protein 16 (VP16), named reverse transcriptional activator (rTA). In the absence of tetracycline, tTA binds to the tet operators to activate transcription from the minimal promoter, whereas in the presence of tetracycline its association and consequently its transcription activation is prevented.

In some aspects, the cell comprising the regulatable promoter further comprises a fusion between a Tet repressor (TetR) and a herpes simplex virus protein 16 (VP16), named transcriptional activator (TA). In the absence of tetracycline, TA does not binds to the tet operators and no transcription from the minimal promoter occurs. In the presence of tetracycline its association and consequently its transcription activation is induced such that the regulatable promoter promotes transcription in opposite direction of the polynucleotides operably linked to each minimal promoter.

In some aspects, the regulatable promoter is located upstream of a first exon of FOXP3 and a first FOXP3 exon is operably linked to one of the minimal promoters of the regulatable promoter. In some aspects, the minimal promoter located on the opposite site of the tet operators relative to the minimal promoter that is operably linked to the FOXP3 first exon is operably linked to a polynucleotide sequence encoding a selectable gene. Therefore, in some aspects, once the tet operators bind tetracycline, the first minimal promoter promotes transcription of the first FOXP3 exon and at the same time the second minimal promoter promotes transcription of the polynucleotide encoding the selectable gene in the opposite direction.

In some aspects, the first minimal promoter promotes transcription of the first FOXP3 exon and at the same time the second minimal promoter promotes transcription of the polynucleotide encoding the selectable gene in the opposite direction in the absence of tetracycline and once the tet operators bind tetracycline, transcription from both minimal promoters ceases.

In some aspects, the minimal promoters located adjacent to the tet operators are distinct promoters. For example, in some aspects, one minimal promoter is a hCMV IE promoter and the second minimal promoter is a 5′ long terminal repeat of a mouse mammary tumor virus (MMTV) (5′ LTR MMTV promoter, see e.g., Hoffman et al. PNAS 93: 5185-90, 1997). In some aspects, one minimal promoter is a hCMV IE promoter and the second minimal promoter is a minimal promoter of human immunodeficiency virus type 1 (HIV-1) promoter (HIV-1 promoter, see, e.g., Baron et al. Nucl. Acids Res. 23: 3605-06, 1995 and Baron et al., Journal of Molecular and Genetic Medicine 2(1): 107-18, 2006). In some aspects, one minimal promoter is 5′ LTR MMTV promoter and the second minimal promoter is a minimal HIV-1 promoter. In some aspects, the regulatable bidirectional promoter is a bidirectional PtetA and PtetR promoter as disclosed by Nguyen et al., Mol. Imaging Biol. 24: 82-92, 2022.

It is noted that any minimal promoter can used in the polynucleotide constructs of the disclosure as long as they promote transcription in opposite directions. It is further noted that any inducible system can be used in the disclosure as long as the inducible element is located between two promoters that promote transcription in opposite directions.

In some aspects, the polynucleotide of the disclosure comprises a recombinant FOXP3 homology donor vector construct.

In some aspects, the recombinant FOXP3 homology donor vector comprises a portion or the entire nucleotide sequence of SEQ ID NO:1 or a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the recombinant FOXP3 homology donor vector is capable of generating a Treg-like cell by transfection of a CD4+T lymphocyte.

In aspects described herein, the FOXP3 donor construct of the disclosure is inserted “downstream” of the TSDR with respect to the TSDR and its location “upstream” of exons 2-12 of the FOXP3 gene. This means that the FOXP3 donor construct is inserted anywhere between the TSDR and exon 2, 3, 4, 5, 6, 7, 8, 9, 10, 11, or 12 of the FOXP3 gene.

In some aspects, the 5′ homology arm comprises a nucleic acid sequence of a FOXP3 exon or portion thereof. In some aspects, the 5′ homology arm comprises a nucleic acid sequence of a FOXP3 intron or portion thereof. In some aspects, the 5′ homology arm comprises a nucleic acid sequence of a FOXP3 T cell-specific demethylation region (TSDR). In some aspects, the 5′ homology arm comprises a nucleic acid sequence of a portion of a FOXP3 intron 1 located between TSDR and exon 2 of the FOXP3 gene.

In some aspects, the 5′ homology arm comprises a nucleic acid sequence of a FOXP3 exon 2 or portion thereof. In some aspects, the 5′ homology arm comprises a nucleic acid sequence of a FOXP3 intron 2 or portion thereof, wherein intron 2 is located between exon 2 and exon 3 of the FOXP3 gene.

In some aspects, the 5′ homology arm comprises a nucleic acid sequence of a FOXP3 exon 3 or portion thereof. In some aspects, the 5′ homology arm comprises a nucleic acid sequence of a FOXP intron 3 or portion thereof, wherein intron 3 is located between exon 3 and exon 4 of the FOXP3 gene.

In some aspects, the 5′ homology arm comprises a nucleic acid sequence of a FOXP3 exon 4 or portion thereof. In some aspects, the 5′ homology arm comprises a nucleic acid sequence of a FOXP3 intron 4, wherein intron 4 is located between exon 4 and exon 5 of the FOXP3 gene.

In some aspects, the 5′ homology arm comprises a nucleic acid sequence of a FOXP3 exon 5 or portion thereof. In some aspects, the 5′ homology arm comprises a nucleic acid sequence of a FOXP3 intron 5, wherein intron 5 is located between exon 5 and exon 6 of the FOXP3 gene.

In some aspects, the 5′ homology arm comprises a nucleic acid sequence of a FOXP3 exon 6 or portion thereof. In some aspects, the 5′ homology arm comprises a nucleic acid sequence of a FOXP3 intron 6, wherein intron 6 is located between exon 6 and exon 7 of the FOXP3 gene.

In some aspects, the 5′ homology arm comprises a nucleic acid sequence of a FOXP3 exon 7 or portion thereof. In some aspects, the 5′ homology arm comprises a nucleic acid sequence of a FOXP3 intron 7, wherein intron 7 is located between exon 7 and exon 8 of the FOXP3 gene.

In some aspects, the 5′ homology arm comprises a nucleic acid sequence of a FOXP3 exon 8 or portion thereof. In some aspects, the 5′ homology arm comprises a nucleic acid sequence of a FOXP3 intron 8, wherein intron 8 is located between exon 8 and exon 9 of the FOXP3 gene.

In some aspects, the 5′ homology arm comprises a nucleic acid sequence of a FOXP3 exon 9 or portion thereof. In some aspects, the 5′ homology arm comprises a nucleic acid sequence of a FOXP3 intron 9, wherein intron 9 is located between exon 9 and exon 10 of the FOXP3 gene.

In some aspects, the 5′ homology arm comprises a nucleic acid sequence of a FOXP3 exon 10 or portion thereof. In some aspects, the 5′ homology arm comprises a nucleic acid sequence of a FOXP3 intron 10, wherein intron 10 is located between exon 10 and exon 11 of the FOXP3 gene.

In some aspects, the 5′ homology arm comprises a nucleic acid sequence of a FOXP3 exon 11 or portion thereof. In some aspects, the 5′ homology arm comprises a nucleic acid sequence of a FOXP3 intron 11, wherein intron 11 is located between exon 11 and exon 12 of the FOXP3 gene.

In some aspects, the 3′ homology arm comprises a nucleic acid sequence of a FOXP3 exon or portion thereof. In some aspects, the 3′ homology arm comprises a nucleic acid sequence of a FOXP3 intron or portion thereof. In some aspects, the 3′ homology arm comprises a nucleic acid sequence of a FOXP3 exon 2 or portion thereof. In some aspects, the 3′ homology arm comprises a nucleic acid sequence of a portion of a FOXP3 intron 1, wherein the portion of the intron 1 is located between the TSDR and exon 2 of the FOXP3 gene.

In some aspects, the 3′ homology arm comprises a nucleic acid sequence of a FOXP3 exon 3 or portion thereof. In some aspects, the 3′ homology arm comprises a nucleic acid sequence of a FOXP3 intron 2, wherein intron 2 is located between exon 2 and exon 3 of the FOXP3 gene.

In some aspects, the 3′ homology arm comprises a nucleic acid sequence of a FOXP3 exon 4 or portion thereof. In some aspects, the 3′ homology arm comprises a nucleic acid sequence of a FOXP3 intron 3, wherein intron 3 is located between exon 3 and exon 4 of the FOXP3 gene.

In some aspects, the 3′ homology arm comprises a nucleic acid sequence of a FOXP3 exon 5 or portion thereof. In some aspects, the 3′ homology arm comprises a nucleic acid sequence of a FOXP3 intron 4, wherein intron 4 is located between exon 4 and exon 5 of the FOXP3 gene.

In some aspects, the 3′ homology arm comprises a nucleic acid sequence of a FOXP3 exon 6 or portion thereof. In some aspects, the 3′ homology arm comprises a nucleic acid sequence of a FOXP3 intron 5, wherein intron 5 is located between exon 5 and exon 6 of the FOXP3 gene.

In some aspects, the 3′ homology arm comprises a nucleic acid sequence of a FOXP3 exon 7 or portion thereof. In some aspects, the 3′ homology arm comprises a nucleic acid sequence of a FOXP3 intron 6, wherein intron 6 is located between exon 6 and exon 7 of the FOXP3 gene.

In some aspects, the 3′ homology arm comprises a nucleic acid sequence of a FOXP3 exon 8 or portion thereof. In some aspects, the 3′ homology arm comprises a nucleic acid sequence of a FOXP3 intron 7, wherein intron 7 is located between exon 7 and exon 8 of the FOXP3 gene.

In some aspects, the 3′ homology arm comprises a nucleic acid sequence of a FOXP3 exon 9 or portion thereof. In some aspects, the 3′ homology arm comprises a nucleic acid sequence of a FOXP3 intron 8, wherein intron 8 is located between exon 8 and exon 9 of the FOXP3 gene.

In some aspects, the 3′ homology arm comprises a nucleic acid sequence of a FOXP3 exon 10 or portion thereof. In some aspects, the 3′ homology arm comprises a nucleic acid sequence of a FOXP3 intron 9, wherein intron 9 is located between exon 9 and exon 10 of the FOXP3 gene.

In some aspects, the 3′ homology arm comprises a nucleic acid sequence of a FOXP3 exon 11 or portion thereof. In some aspects, the 3′ homology arm comprises a nucleic acid sequence of a FOXP3 intron 10, wherein intron 10 is located between exon 10 and exon 11 of the FOXP3 gene.

In some aspects, the 3′ homology arm comprises a nucleic acid sequence of a FOXP3 exon 12 or portion thereof. In some aspects, the 3′ homology arm comprises a nucleic acid sequence of a FOXP3 intron 11, wherein intron 11 is located between exon 11 and exon 12 of the FOXP3 gene.

In some aspects, the FOXP3 nucleic acid and protein sequences may be derived from any source. A number of FOXP3 nucleic acid and protein sequences are known. A representative example of a human FOXP3 sequence is presented in NCBI entry NG_007392, and additional representative sequences including various isoforms of the FOXP3 transcription factor are listed in the National Center for Biotechnology Information (NCBI) database, which sequences are herein incorporated by reference (see also, for example, NCBI entries: Accession Nos. NM_001114377, and NM_014009). Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a FOXP3 homology donor construct, wherein the expressed variant FOXP3 retains biological activity, including transcription factor activity and the ability to convert CD4+T lymphocytes, NK T cells, or HSPC into engineered FOXP3 T cells.

In some aspects, the selectable marker is a cell surface marker gene for in vitro selection and in vivo tracking of cells transduced with the FOXP3 donor vector.

In some aspects, the selectable marker gene is selected from a polynucleotide encoding a B cell surface protein, or a neuronal cell surface protein.

In some aspects, the B cell surface protein is a CD19, CD22, or CD123 protein. In some aspects, the selectable marker is a polynucleotide encoding a truncated CD19, CD22, or CD123 protein, wherein the truncated CD19, CD22, or CD123 protein lacks an intracellular signaling domain.

In some aspects, the neuronal cell surface protein is an olfactory receptor. In some aspects, the olfactory receptor is Olfactory receptor 2M5, Olfactory receptor 2A25, Olfactory receptor 6C74, Olfactory receptor 1411, Olfactory receptor 5H15, Olfactory receptor 6C68, Olfactory receptor 5K3, Olfactory receptor 6C6, Olfactory receptor 51F1, Olfactory receptor 2T8, Olfactory receptor 4C46, Olfactory receptor 5H14, Olfactory receptor 5H1, Olfactory receptor 6C75, Olfactory receptor 5B21, Olfactory receptor 2AG2, Olfactory receptor 6C76, Olfactory receptor 5K4, Olfactory receptor 4C45, Olfactory receptor 2AT4, or Olfactory receptor 7C2.

In some aspects, the selectable gene is a truncated nerve growth factor receptor gene as described by Fehse et al. Human Gene Therapy 8(15): 1815-24, 2008.

In some aspects, the cell surface marker is a low-affinity nerve growth factor receptor (LNGFR). In some aspects, the LNGFR lacks intracellular signaling components and is referred to herein as a low-affinity, truncated NGFR. In some aspects, the LNGFR comprises a wild-type LNGFR extracellular domain comprising the four TNFR cysteine-rich NGFR domains and the serine/threonine-rich stalk. In some aspects, the LNGFR comprises only the four TNFR cysteine-rich domains. In some aspects, the LNGFR comprises a mutated long LNGFR construct comprising the four TNFR cysteine-rich domains and the stalk, but the fourth domain largely deleted to avoid NGF signaling (Yan H, Chao M V. Disruption of cysteine-rich repeats of the p75 nerve growth factor receptor leads to loss of ligand binding. J Biol Chem (1991) 266(18):12099-104). In some aspects, the LNGFR comprises a mutated short LNGFR comprising only the four TNFR cysteine-rich domains with the mutated version of the fourth domain.

In some aspects, the FOXP3 donor construct comprises a polynucleotide that encodes a therapeutic protein. In some aspects, the polynucleotide encoding the therapeutic protein is under the control of the bidirectional promoter. In some aspects, the polynucleotide that encodes the therapeutic protein is under the control of a separate promoter.

In some aspects, the therapeutic protein is a protein that can treat an autoimmune disorder or condition. In some aspects, the therapeutic protein is a protein that can prevent or ameliorate an autoimmune disease or condition. In some aspects, the therapeutic protein is a protein that can treat an allo-transplant rejection. In some aspects, the therapeutic protein is a protein that can prevent or ameliorate an allo-transplant rejection. In some aspects, the therapeutic protein is a protein that can treat a xeno-transplant rejection. In some aspects, the therapeutic protein is a protein that can prevent or ameliorate a xeno-transplant rejection.

In some aspects, the therapeutic protein is a cytokine. In some aspects, the cytokine is an inhibitory cytokine. In some aspects, the inhibitory cytokine is interleukin-10, interleukin-35 or TGF-β. In some aspects, the therapeutic protein is Granzyme B. In some aspects, the therapeutic protein is indolamine-2,3-dioxygenase. In some aspects, the protein is an anti-IL-2 antibody or fragment thereof. In some aspects, the therapeutic protein is a self-antigen that is involved in the development and/or maintenance of an autoimmune disease or disorder. In some aspects, the therapeutic protein is an antigen that is involved in the development and/or maintenance of an allo-transplant rejection. In some aspects, the therapeutic protein is an antigen that is involved in the development and/or maintenance of a xeno-transplant rejection.

In some aspects, the polynucleotide encoding the cell surface marker comprises a polyadenylation (polyA) site. In some aspects, the poly A is a SV40 polyA.

The polynucleotides disclosed herein can further comprise one or more inverted terminal repeats (ITRs). In some aspects, the polynucleotide comprises a first ITR and a second ITR. In some aspects, the polynucleotide comprises a first ITR, e.g., a 5′ ITR, and a second ITR, e.g., a 3′ ITR. Typically, ITRs are involved in parvovirus (e.g., adeno-associated virus (AAV)) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. In some aspects, the ITRs fold into a hairpin T-shaped structure. In some aspects, the ITRs fold into non-T-shaped hairpin structures, e.g., into a U-shaped hairpin structure.

In some aspects, the ITRs that are useful for the present disclosure comprise an ITR from an AAV genome. In certain aspects, the ITR is an ITR of an AAV genome selected from AAV1, AAV2, AAV3, AAV4, AAV5, AAV6, AAV7, AAV8, AAV9, AAV10, AAV11, and any combination thereof. In some aspects, the ITR is an ITR of the AAV2 genome. In some aspects, the ITR is a synthetic sequence genetically engineered to include at its 5′ and 3′ ends ITRs derived from one or more of AAV genomes.

In some aspects, the ITR is not derived from an AAV genome. In some aspects, the ITR is an ITR of a non-AAV. In some aspects, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain aspects, the ITR is derived from erythrovirus parvovirus B19 (human virus). In some aspects, the ITR is derived from a Muscovy duck parvovirus (MDPV) strain. In certain aspects, the MDPV strain is attenuated, e.g., MDPV strain FZ91-30. In some aspects, the MDPV strain is pathogenic, e.g., MDPV strain YY. In some aspects, the ITR is derived from a porcine parvovirus, e.g., porcine parvovirus U44978. In some aspects, the ITR is derived from a mice minute virus, e.g., mice minute virus U34256. In some aspects, the ITR is derived from a canine parvovirus, e.g., canine parvovirus M19296. In some aspects, the ITR is derived from a mink enteritis virus, e.g., mink enteritis virus D00765. In some aspects, the ITR is derived from a Dependoparvovirus. In certain aspects, the Dependoparvovirus is a Dependovirus Goose parvovirus (GPV) strain. In some aspects, the GPV strain is attenuated, e.g., GPV strain 82-0321V. In some aspects, the GPV strain is pathogenic, e.g., GPV strain.

The polynucleotides disclosed herein can also comprise a mammalian origin of replication (e.g., an Epstein Barr virus origin of replication) in order to maintain the vector extrachromosomally and produce multiple copies of the vector in a cell.

In some aspects, the 3′ UTR poly(A) tail sequence is a 3′ UTR SV40 poly(A) tail sequence (SEQ ID NO: XX), a 3′ UTR bovine growth hormone (bGH) poly(A) sequence (SEQ ID NO: XX), a 3′ UTR actin poly(A) tail sequence, a 3′ UTR hemoglobin poly(A) sequence, LTR poly(A) tail, human growth hormone (hGH) poly(A) tail, or human (3-globin poly(A) tail or any combinations thereof.

In some aspects of the disclosure, a TALEN gene editing system is used to generate a FOXP3 expressing cell. TALEN based genome editing methods provide advantages of targeted introduction of a promoter of choice in the endogenous FOXP3 gene and regulatable or constitutive expression of FOXP3 in gene edited cells.

In some aspects of the disclosure, a Zinc-Finger Nuclease gene editing system is used to generate a FOXP3 expressing cell.

In some aspects of the disclosure, a Meganuclease gene editing system is used to generate a FOXP3 expressing cell.

In some aspects of the disclosure, a Restriction Endonuclease gene editing system is used to generate a FOXP3 expressing cell.

### Polynucleotide Delivery

The polynucleotides described above can be delivered using viral vectors. In some aspects, the polynucleotides are provided in a viral vector. In some aspects, the viral vectors comprising the polynucleotides described herein comprise additional DNA segments that are portions of the genome of the viral vectors used and aid in generation of viral particles for the delivery of the polynucleotides described herein. In some aspects the viral vectors include, but are not limited to: an adenoviral vector, an adeno-associated virus (AAV) vector, retroviral vector, a lentiviral vector, poxvirus vector, a baculovirus vector, a herpes viral vector, simian virus 40 (SV40), cytomegalovirus (CMV), mouse mammary tumor virus (MMTV), and Moloney murine leukemia virus.

In some aspects, the polynucleotides described herein are delivered to a cell using non-viral delivery methods. In some aspects, the non-viral delivery comprises plasmid electroporation. In some aspects, the non-viral delivery comprises a lipid. In some aspects, the lipid is a lipid vesicle. In some aspects, the lipid vesicle is a micelle, a liposome, a lipid nanoparticle, or an extracellular vesicle. In some aspects, a polynucleotide described herein is delivered to a cell using electroporation. In some aspects, a protein described herein is delivered to a cell using electroporation. In some aspects, a polynucleotide described herein is delivered to a cell using a lipid vesicle. In some aspects, a protein described herein is delivered to a cell using a lipid vesicle.

In some aspects, more than one delivery method is used together or in sequence to deliver the polynucleotides and/or protein described herein to a cell. For example, in some aspects, the polynucleotide comprising the FOXP3 donor construct can be delivered in a viral vector to a cell and the nuclease protein can be delivered using a lipid vesicle or electroporation. In some aspects, the polynucleotides comprising the FOXP3 donor construct can be delivered using a plasmid and electroporation and a plasmid or a lipid vesicle and the nuclease can be delivered using a viral vector, a lipid vesicle, or electroporation and a plasmid or a nuclease protein.

### Compositions

The present disclosure provides pharmaceutical compositions comprising the polynucleotides of the disclosure, and at least one pharmaceutically acceptable excipient.

In some aspects, the present disclosure provides gene therapy compositions comprising the polynucleotides of the disclosure, and at least one pharmaceutically acceptable excipient.

In some aspects the compositions comprise a FOXP3 donor polynucleotide construct, vector or nuclease described herein in a physiologically acceptable carrier, excipient or stabilizer. The acceptable carriers, excipients, or stabilizers are nontoxic to recipients at the dosages and concentrations employed.

A carrier can be a diluent, adjuvant, excipient, or vehicle with which the FOXP3 donor construct, vector or nuclease is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intraarticularly and the like) the active compound may be coated in a material to protect the compound from the action of acids and other natural conditions that may inactivate the compound. For example, a pharmaceutical composition can be formulated for parenteral, e.g., intravenous, administration. The compositions to be used for in vivo administration can be sterile. This is readily accomplished by filtration through, e.g., sterile filtration membranes. The pharmaceutical compositions described herein are in one aspect for use as a medicament.

In some aspects, the composition of the present disclosure comprise adjuvants such as preservatives, wetting agents, emulsifying agents and dispersing agents. The presence of microorganisms can be avoided by the sterilization procedure described above or together with the inclusion of various antibacterial and antifungal agents, for example, parabens, chlorobutanol, phenol, sorbic acid, and the like. It may also be desirable to include isotonic agents, such as sugars, sodium chloride, and the like, in the composition. In addition, prolonged absorption of the injectable pharmaceutical form may be brought about by the inclusion of an agent, which delays absorption, such as aluminum monostearate and gelatin.

### Methods of Making of Polynucleotides

Provided is a method of making a polynucleotide for expression of FOXP3. In some aspects, the method comprises providing a first nucleotide sequence that comprises a coding strand and a targeted locus, where the coding strand comprises one or more regulatory elements and a FOXP3 gene, and the targeted locus comprises an intron sequence of the FOXP3 gene. In some aspects, the method further comprises providing a second nucleic acid sequence. In some aspects, the method further comprises providing a nuclease. In some aspects, the method further comprises performing a gene editing process on the first nucleotide sequence, which edits said intron sequence, and inserts the second nucleic acid into the targeted locus. In some aspects, the insertion of the second nucleic acid results in expression of FOXP3.

In some aspects, the targeted locus is a genome locus. In some aspects, the second nucleotide sequence comprises a heterologous promoter operably linked to a polynucleotide comprising at least one FOXP3 exon or a portion thereof. In some aspects, the second nucleotide sequence further comprises a polynucleotide encoding a selectable marker protein.

In some aspects, the heterologous promoter is a bidirectional promoter. In some aspects, when the second nucleic acid is inserted in the targeted locus, the promoter controls transcription of the polynucleotide comprising the at least one FOXP3 exon or portion thereof and the polynucleotide encoding the selectable marker protein. In some aspects, when the second nucleic acid is inserted in the targeted locus, the bidirectional promoter promotes transcription of the polynucleotide comprising the at least one FOXP3 exon or portion thereof in the direction of the additional exons of the FOXP3 gene and the polynucleotide encoding the selectable marker protein in the opposite direction, e.g., in the direction of the location of the TSDR.

In some aspects, the promoter is a heterologous promoter. In some aspects, the heterologous promoter is a bidirectional promoter that controls transcription of the polynucleotide comprising the at least one FOXP3 exon or portion thereof and the polynucleotide encoding the selectable marker protein in opposite directions.

In some aspects, the selectable marker protein is a cell surface protein. In some aspects, the selectable marker protein is a truncated, low-affinity nerve growth factor receptor protein.

In some aspects, the second nucleotide sequence comprises exons 1, 2, and 3 of FOXP3 and the targeted locus is at an intron between exon 2 and exon 3 of the FOXP3 gene of the first nucleotide sequence.

In some aspects, the second nucleotide sequence comprises exons 1, 2, 3, and 4 of FOXP3 and the targeted locus is at an intron between exon 3 and exon 4 of the FOXP3 gene of the first nucleotide sequence.

### Methods of Use

The present disclosure also provides methods of stimulating T suppressor function or inhibiting an immune response in a subject, the method comprising administering an effective amount of a cell comprising a FOXP3 donor construct disclosed herein to the subject. Also provided are methods of suppressing responder T cell activation by non-human cells, e.g., porcine cells in a subject comprising non-human cells, the method comprising administering an effective amount of a cell comprising a FOXP3 donor construct disclosed herein to the subject. Further provided are methods of suppressing responder T cell activation by unmatched human donor cells in a subject comprising unmatched human donor cells, the method comprising administering an effective amount of a cell comprising a FOXP3 donor construct disclosed herein to the subject.

The present disclosure also provides methods to generate a cell for therapy comprising inserting a FOXP3 donor construct comprising a nucleic acid encoding portions of or an entire FOXP3 gene under the control of an exogenous promoter into the cell, wherein insertion of the FOXP3 donor construct activates expression of the FOXP3 gene. In some aspects, the cell that receives the FOXP3 donor construct is a T cell, a NK T cell or a HSPC. The present disclosure provides a method of generating a persisting population of genetically engineered cells in a subject, the method comprising administering to the subject a cell genetically engineered to express a FOXP3 donor construct polynucleotide disclosed herein. In some aspects, the method comprises culturing the cell under suitable conditions. In some aspects, the method comprises expanding the cell under suitable conditions before administering the cell to a subject.

In some aspects, the polynucleotides comprising a FOXP3 donor construct are used to transduce a hematopoietic cell. In some aspects, the hematopoietic cell is a hematopoietic stem cell. In some aspects, the hematopoietic cell is a hematopoietic progenitor cell. In some aspects, the hematopoietic cell is a NK T cell. In some aspects, the hematopoietic cell is a lymphocyte. In some aspects, the lymphocyte is a T lymphocyte. In some aspects, the T lymphocyte is a CD4+T lymphocyte. In some aspects, the T lymphocyte is a Treg cell.

In some aspects, the hematopoietic cell is optionally purified before or after gene editing by any method known in the art including, but not limited to, density gradient centrifugation (e.g., Ficoll Hypaque, percoll, iodoxanol and sodium metrizoate), immunoselection (positive selection or negative selection for surface markers) with immunomagnetic beads or immunoaffinity columns, or fluorescence-activated cell sorting (FACS). For example, CD4+T lymphocytes, CD34+ HSPC, or NK T cells can be isolated from apheresis products by immune-magnetic cell selection, cultured in the presence of IL-2 and IL-7, then transfected or transduced with a FOXP3 homology donor vector, followed by immunoselection for the cell surface marker (e.g., LNGFR) expressed by the recombinant FOXP3 homology donor vector to separate gene edited cells from non-gene edited cells.

In some aspects, the hematopoietic cell is a hematopoietic stem cell and is obtained by harvesting from bone marrow, from peripheral blood or cord blood of a subject. In some aspects, bone marrow is aspirated from the posterior iliac crests while the donor is under either regional or general anesthesia. Additional bone marrow can be obtained from the anterior iliac crest. In some aspects, a dose of 1×10⁸ to 2×10⁸ marrow mononuclear cells per kilogram is considered a desirable dose to establish engraftment in an autologous and/or allogeneic marrow transplant. In some aspects, the bone marrow is primed with granulocyte colony-stimulating factor (G-CSF; filgrastim [Neupogen]) to increase the stem cell count.

In some aspects, the stem cells of a subject are mobilized from the bone marrow into peripheral blood by administration of cytokines such as G-CSF or GM-CSF and peripheral blood progenitor cells are collected by apheresis. In some aspects, the dose of G-CSF used for mobilization from about 10 μg/kg/day to about 40 μg/kg/day can be given. In some aspects, Mozobil® is used in conjunction with G-CSF to mobilize hematopoietic stem cells to peripheral blood for collection. In some aspects, the stem cells are purified. In some aspects, unpurified stem cells are used according to the methods disclosed herein. Stem cell purification methods include, but are not limited to, flow cytometry; an isolex system (See, e.g., Klein et al. (2001) Bone Marrow Transplant. 28(11):1023-9; Prince et al. (2002) Cytotherapy 4(2):137-45); immunomagnetic separation (Prince et al. (2002) Cytotherapy 4(2): 147-55; Handgretinger et al. (2002) Bone Marrow Transplant. 29(9):731-6; Chou et al. (2005) Breast Cancer. 12(3):178-88); and the like. Each of these references is herein specifically incorporated by reference, particularly with respect to procedures, cell compositions and doses for hematopoietic stem and progenitor cell transplantation.

In some aspects, the minimum dose infused into a recipient for engraftment is 1-2×10⁶ CD34+ cells/kg body weight for autologous and/or allogeneic transplants. The cells which are employed may be fresh, frozen, or have been subject to prior culture. They may be fetal, neonate, adult, etc. Hematopoietic stem cells may be obtained from fetal liver, bone marrow, cord blood, peripheral blood, particularly G-CSF or GM-CSF mobilized peripheral blood, or any other conventional source. Cells for engraftment are optionally isolated from other cells, where the manner in which the stem cells are separated from other cells of the hematopoietic or other lineage is not critical to the disclosure.

The ability of the resulting engineered FOXP3-expressing cells to respond to activation or to suppress proliferation and activation of effector T cells and other immune cells can be assayed by methods disclosed herein as well as by methods known in the art including, for example, without limitation, performing an in vitro suppression assay or 3H-thymidine assay that measures suppression of T cell proliferation by engineered FOXP3-expressing T cells, or a flow cytometry-based suppression assay that measures suppression of proliferation and cytokine production in subpopulations of T cells and other immune cells (see, e.g., Thornton et al. (1998) J. Exp. Med. 1998. 188:287-296, Schneider et al. (2011) Methods Mal. Biol. 707:233-241, Baecher-Allan et al. (2005) Clin. Immunol. 115:10-18, McMurchy et al. (2012) Eur. J. Immunol. 42:27-34; herein incorporated by reference.

In some aspects, the production of FOXP3 in cells transduced with a FOXP3 donor vector is assessed, for example, by using a real-time RT-PCR assay of FOXP3 mRNA levels or a Western Blot assay of FOXP3 protein levels. In some aspects, the ability of the CRISPR/Cas9 and FOXP3 homology donor vector to confer physiologic Treg characteristics on CD4+T lymphocytes is evaluated in vitro using a proliferation or a suppression assay as disclosed herein.

In some aspects, provided is a use of a polynucleotide comprising a FOXP3 donor construct as described herein in the manufacture of a medicament for treating a subject.

In some aspects, provided is a use of a polynucleotide comprising a FOXP3 donor construct as described herein for treating a subject.

### Indications

The present disclosure provides methods of treating an auto-immune disease or condition, allo-transplant rejection or xeno-transplant rejection in a subject in need thereof comprising administering to the subject a therapeutically effective amount of a composition, pharmaceutical composition, polynucleotide, system or cell as described herein. The present disclosure also provides methods of preventing or ameliorating the symptoms of an auto-immune disease or condition, allo-transplant rejection or xeno-transplant rejection in a subject in need thereof comprising administering to the subject a therapeutically effective amount of a composition, pharmaceutical composition, polynucleotide, system or cell as described herein. In some aspects, the compositions disclosed herein (e.g., polynucleotides encoding a FOXP3 donor construct of the present disclosure, vectors comprising polynucleotides encoding FOXP3 donor constructs of the present disclosure, FOXP3 donor constructs of the present disclosure, or cells expressing FOXP3 donor constructs of the present disclosure) can be used to treat a disease or condition, e.g., an auto-immune disease, an allo-transplant rejection, a xeno-transplant rejection or any disease or condition in which suppression of an immune response affords a prophylactic or therapeutic effect. Allo-transplant recipients to be treated using a composition described herein include recipients of any organ or tissue that can be allo-transplanted, that is, transplanted between genetically non-identical individuals of the same species. Xeno-transplant recipients to be treated using a composition described herein include recipients of any organ or tissue that can be xeno-transplanted, that is, transplanted from one species to another. For example, in some aspects, the xeno-transplant recipient treated according to methods disclosed herein is a recipient of a kidney, a heart, lungs, or another organ or tissue of a species different from the recipient's species.

Auto-immune diseases or conditions are known to a person skilled in the art and can be treated using a method disclosed herein irrespective of the origin or source of auto-immunity. In some aspects, the diseases or disorders treated with a composition, pharmaceutical composition, polynucleotide, system or cell described herein include Lupus nephritis, anti-glomerular basement membrane nephritis, auto-immune hepatitis, primary biliary cholangitis, primary sclerosing cholangitis, anti-synthethase syndrome, interstitial cystitis, myocarditis, post-myocardial infarction syndrome, subacute bacterial endocarditis, alopecia areata, autoimmune angioedema, auto-immune urticaria, bullous pemphigus, dermatitis herpetiformis, discoid lupus erythematosus, epidermolysis bullosa acquisita, erythema nodosum, gestational pemphigoid, lichen planus, lichen sclerosus, linear IgA disease, morphea, pemphigus vulgaris, psoriasis, systemic scleroderma, vitiligo, Addison's disease, auto-immune polyendocrine syndrome types 1-3, autoimmune pancreatitis, diabetes mellitus type 1, autoimmune thyroiditis, Grave's disease, endometriosis, autoimmune orchitis, Sjoergen syndrome, autoimmune enteropathy, coeliac disease, Crohn's disease, esophageal achalasia, ulcerative colitis, anti-phospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evan's syndrome, pernicious anemia, pure red cell aplasia, thrombocytopenia, adiposis dolorosa, adult-onset Still's syndrome, ankylosing spondylitis, CREST syndrome, drug-induced lupus, enthesitis-related arthritis, eosinophilic fascitis, juvenile arthritis, chronic Lyme disease, mixed connective tissue disease, palindromic rheumatism, Parry-Romberg syndrome, Parsonage-Turner syndrome, psoriatic arthritis, relapsing polychondritis, retroperitoneal fibrosis, rheumatic fever, rheumatic arthritis, sarcoidosis, Schnitzler syndrome, Systemic lupus erythematosus, dermatomyositis, fibromyalgia, inclusion body myositis, myasthenia gravis, neuromyotonia, paraneoplastic cerebellar degeneration, polymyositis, acute disseminating encephalomyelitis, acute motor axonal neuropathy, anti-NMDA receptor encephalitis, chronic inflammatory demyelinating polyneuropathy, Guillain-Barre syndrome, Hashimoto encephalitis, idiopathic inflammatory demyelinating disease, Lambert-Eaton myasthenia syndrome, multiple sclerosis, progressive inflammatory neuropathy, restless legs syndrome, transverse myelitis, autoimmune retinopathy, autoimmune uveitis, Cogan syndrome, Grave's ophthalmopathy, intermediate uveitis, ligneous conjunctivitis, Mooren's ulcer, neuromyelitis optica, opsoclonus myoclonus syndrome, optic neuritis, scleritis, sympathetic ophthalmia, autoimmune inner ear disease, Behcet's syndrome, eosinophilic granulomatosis with polyangitis, giant cell arteritis, IgA vasculitis, Kawasaki disease, leukocytoclastic vasculitis, lupus vasculitis, rheumatoid vasculitis, polyarteritis nodosa, polymyalgia rheumatica, or urticarial vasculitis.

### Kits and Articles of Manufacture

The disclosure also provides a kit comprising (i) a cell genetically modified to express a FOXP3 donor construct of the present disclosure, i.e., a cell comprising one or more polynucleotides encoding a FOXP3 donor construct of the present disclosure, or one or more vectors encoding a FOXP3 donor construct of the present disclosure (e.g., a hematopoietic cell, preferably a T cell), or a pharmaceutical composition comprising the cell, and optionally (ii) instructions for use. Also provided is an article of manufacture comprising a cell genetically modified to express a FOXP3 donor construct described herein, one or more polynucleotides encoding a FOXP3 donor construct described herein, or a pharmaceutical composition as described herein.

In some aspects, the kit or article of manufacture comprises at least a polynucleotide or vector encoding a FOXP3 donor construct of the present disclosure, a cell genetically modified to express a FOXP3 donor construct of the present disclosure, or a composition (e.g., a pharmaceutical composition) comprising a polynucleotide, vector, or cell disclosed herein, in one or more containers.

In some aspects, the kit or article of manufacture comprises at least a polynucleotide or vector encoding a FOXP3 donor construct of the present disclosure, a cell genetically modified to express a FOXP3 donor construct of the present disclosure, or a composition (e.g., a pharmaceutical composition) comprising a polynucleotide, vector, or cell disclosed herein, and optionally a brochure.

One skilled in the art will readily recognize that the polynucleotides, vectors, cells, and compositions of the present disclosure, pharmaceutical composition comprising the polynucleotides, vectors, or cells of the present disclosure, or combinations thereof can be readily incorporated into one of the established kit formats which are well known in the art.

In some aspects, the kit or article of manufacture comprises, e.g., a polynucleotide or vector encoding a FOXP3 donor construct of the present disclosure, or a composition (e.g., a pharmaceutical composition) comprising a polynucleotide, vector, in dry form in a container (e.g., a glass vial), and optionally a vial with a solvent.

In some aspects, the kit or article of manufacture comprises, e.g., a polynucleotide or vector encoding a FOXP3 donor construct of the present disclosure, or a composition (e.g., a pharmaceutical composition) comprising a polynucleotide, vector, in at least one container, and another or more containers with transfection reagents.

In some aspects, the present disclosure provides a kit or article of manufacture comprising a gRNA for CRISPR/Cas9 mediated insertion in the FOXP3 gene wherein the gRNA is directed to the recognition/insertion site ATCCACCGTTGAGAGCTGGG (SEQ ID NO: 1).

### EXAMPLES

### Example 1—Construct Preparation

The DNA sequences of the FOXP3 gene comprising nucleotides 6,631 to 7,130 (SEQ ID NO: 2) and 7,131 to 7,630 (SEQ ID NO: 3) as found in PubMed Accession number 50943 with the exception of a C to G mutation at site 7,121 were de novo synthesized using a BioXP 3200. The sequences corresponding to FOXP3 exons 1, 2, 3, and 4 (SEQ ID NO: 4, 5, 6 and 7) were de novo synthesized and attached to the 3′ end of the synthesis corresponding to the sequence of nucleotides 6631 to 7130. The sequences for the bi-directional PGK promoter (SEQ ID NO: 8) or a bidirectional ubiquitin promoter (UbC) (SEQ ID NO: 10), which both comprise a mini cytomegalovirus (CMV) promoter (SEQ ID NO: 9), a low affinity nerve growth factor receptor gene lacking the intracellular signaling domain (NGFR) (SEQ ID NO: 11), and a SV40poly(A) signal (SEQ ID NO: 12) were PCR amplified and the PGK/mini CMV and UbC/mini CMV promoters, respectively, were cloned between the FOXP3 exons and the NGFR-SV40-poly(A) such that the NGFR-SV40-polyA was transcribed in the opposite direction of the FOXP3 exonic sequences. This large polynucleotide was cloned into a plasmid containing AAV2 inverted terminal repeats (SEQ ID NO: 13 and 14) obtained from Cell Biolabs Inc. (https://www.cellbiolabs.com/aav-expression-and-packaging) to replace the polynucleotide framed by the AAV2 to create the pAAV.FOXP3.PGKProm.NGFR and pAAV.FOXP3.UbCProm.NGFR constructs. Recombinant AAV6.FOXP3 vectors were generated using the AAV.FOXP3.NGFR polynucleotides and a AAV2-Rep-AAV6-Cap plasmid and an Ad helper plasmid (obtained from Cell Biolabs Inc.) in HEK293 cells. Furthermore, FOXP3 homology arms were inserted between the AAV ITRs and the SV40 poly and FOXP3 exon 4, respectively. Thus, the FOXP3.PGKprom.NGFR-ITR polynucleotide (SEQ ID NO: 15) comprises the AAV-ITRs, a PGK/mini CMV promoter located between a delta-LNGFR gene and the FOXP3 exons 1-4, a SV40 poly downstream of the delta-LNGFR gene and one FOXP3 homology arm between the SV40 poly and one AAV ITR and the other FOXP3 homology arm between FOXP3 exon 4 and the other AAV ITR. The FOXP3.UbCprom.NGFR-ITR polynucleotide (SEQ ID NO: 16) comprises the AAV-ITRs, a Ubiquitin C/mini CMV promoter located between a delta-LNGFR gene and the FOXP3 exons 1-4, a SV40 poly downstream of the delta-LNGFR gene and one FOXP3 homology arm between the SV40 poly and one AAV ITR and the other FOXP3 homology arm between FOXP3 exon 4 and the other AAV ITR.

TABLE 1

| SEQ | | |
|---|---|---|
| ID NO. | Name | sequence |
| 1 | CRISPR target sequence | ATCCACCGTTGAGAGCTGGG |
| 2 | FOXP3 5′ homology arm | agaggaggaggccgtattgtagtcccatgagcatagctatgtgtccccatcccc |
| | | atgtgacaagagaagaggactggggccaagtaggtgaggtgacagggctga |
| | | ggccagctctgcaacttattagctgtttgatctttaaaaagttactcgatctccatga |
| | | gcctcagtttccatacgtgtaaaagggggatgatcatagcatctaccatgtgggc |
| | | ttgcagtgcagagtatttgaattagacacagaacagtgaggatcaggatggcct |
| | | ctcacccacctgcctttctgcccagctgcccacactgcccctagtcatggtggca |
| | | ccctccggggcacggctgggccccttgccccacttacaggcactcctccagga |
| | | caggccacatttcatgcaccaggtatggacggtgaatgggcagggaggaggg |
| | | agcaggtgggagaactgtggggaggggccccgagtcaggctgaaccacagc |
| | | ccacatgtgccccccag |
| 3 | FOXP3 3′ homology arm | ctctcaacggtggatgcccacgcccggacccctgtgctgcaggtgcaccccct |
| | | ggagagcccagccatgatcagcctcacaccacccaccaccgccactggggtc |
| | | ttctccctcaaggcccggcctggcctcccacctggtaacacctcagcccgtacc |
| | | ccatggcttcacagaacccccaagtccccagatccttggctgtgagcagtgtag |
| | | gctattctgaattgcagtactctgggggtcaaaggtgtcaggtctcagaggcttg |
| | | gaaactccaccctccaaaaaacgtcaggtgcagaaccttaaagatgcagaatgt |
| | | caaaatcacaaaaccacagagctttacaaagctagtcaaaatgtcagcacctgc |
| | | gaatggccgtctttaagcttctctgccagaagcctgggactttggggacagcag |
| | | agccccctgggagtcagggttttcgaggctcaggagggtgggaagctcaaaat |
| | | gagaggccttgtgggcc |
| 4 | FOXP3 exon 1 | gcacacactcatcgaaaaaaatttggattattagaagagagaggtctgcggcttc |
| | | cacaccgtacagcgtggtttttcttctcggtataaaagcaaagttgtttttgatacgt |
| | | gacagtttcccacaagccaggctgatccttttctgtcagtccacttcaccaag |
| 5 | FOXP3 exon 2 | cctgcccttggacaaggacccgatgcccaaccccaggcctggcaagccctcg |
| | | gccccttccttggcccttggcccatccccaggagcctcgcccagctggagggc |
| | | tgcacccaaagcctcagacctgctgggggcccggggcccagggggaacctt |
| | | ccagggccgagatcttcgaggcggggcccatgcctcctcttcttccttgaaccc |
| | | catgccaccatcgcagctgca |
| 6 | FOXP3 exon 3 | gctgcccacactgcccctagtcatggtggcaccctccggggcacggctgggc |
| | | cccttgccccacttacaggcactcctccaggacaggccacatttcatgcaccag |
| 7 | FOXP3 exon 4 | ctctcaacggtggatgcccacgcccggacccctgtgctgcaggtgcaccccct |
| | | ggagagcccagccatgatcagcctcacaccacccaccaccgccactggggtc |
| | | ttctccctcaaggcccggcctggcctcccacctg |
| 8 | PGK promoter | aattctaccgggtaggggaggcgcttttcccaaggcagtctggagcatgcgctt |
| | | tagcagccccgctgggcacttggcgctacacaagtggcctctggcctcgcaca |
| | | cattccacatccaccggtaggcgccaaccggctccgttctttggtggccccttcg |
| | | cgccaccttctactcctcccctagtcaggaagttcccccccgccccgcagctg |
| | | cgtcgtgcaggacgtgacaaatggaagtagcacgtctcactagtctcgtgcaga |
| | | tggacagcaccgctgagcaatggaagcgggtaggcctttggggcagcggcc |
| | | aatagcagctttgctccttcgctttctgggctcagaggctgggaaggggtgggt |
| | | ccgggggcgggctcaggggcgggctcaggggcggggcgggcgcccgaa |
| | | ggtcctccggaggcccggcattctgcacgcttcaaaagcgcacgtctgccgcg |
| | | ctgttctcctcttcctcatctccgggcctttcgacct |
| 9 | Mini CMV promoter | tatgtcgaggtggcgtgtacggtgggaggcctatataagcagagctcgtttagt |
| | | gaaccgtcagatcg |
| 10 | Ubiquitin C promoter | ccgcggctaaggaacgcgggccgcccacccgctccgggtgcagcggcctcc |
| | | gcgccgggttttggcgcctcccgcgggcgcccccctcctcacggcgagcgct |
| | | gccacgtcagacgaagggcgcagcgagcgtcctgatccttccgcccggacgc |
| | | tcaggacagcggcccgctgctcataagactcggccttagaaccccagtatcag |
| | | cagaaggacattttaggacgggacttgggtgactctagggcactggttttctttcc |
| | | agagagcggaacaggcgaggaaaagtagtcccttctcggcgattctgcggag |
| | | ggatctccgtggggcggtgaacgccgatgattatataaggacgcgccgggtgt |
| | | ggcacagctagttccgtcgcagccgggatttgggtcgcagttcttgtttgtggat |
| | | cgctgtgatcgtcacttagtgagtagcgggctgctgggctggccggggctttcg |
| | | tggccgccgggccgctcggtgggacggaagcgtgtggagagaccgccaag |
| | | ggctgtagtctgggtccgcgagcaaggttgccctgaactgggggttgggggg |
| | | agcgcagcaaaatggcggctgttcccgagtcttgaatggaagacgcttgtgag |
| | | gcgggctgtgaggtcgttgaaacaaggtggagggcatggtgggcggcaaga |
| | | acccaaggtcttgaggccttcgctaatgcgggaaagctcttattcgggtgagat |
| | | gggctggggcaccatctggggaccctgacgtgaagtttgtcactgactggaga |
| | | actcggtttgtcgtctgttgcgggggcggcagttatggcggtgccgttgggcag |
| | | tgcacccgtacctttgggagcgcgcgccctcgtcgtgtcgtgacgtcacccgtt |
| | | ctgttggcttataatgcagggtggggccacctgccggtaggtgtgcggtaggct |
| | | tttctccgtcgcaggacgcagggttcgggcctagggtaggctctcctgaatcga |
| | | caggcgccggacctctggtgaggggagggataagtgaggcgtcagtttctttg |
| | | gtcggttttatgtacctatcttcttaagtagctgaagctccggttttgaactatgcgc |
| | | tcggggttggcgagtgtgttttgtgaagttttttaggcaccttttgaaatgtaatcatt |
| | | tgggtcaatatgtaattttcagtgttagactagtaaattgtccgctaaattctggccg |
| | | tttttggcttttttgttagac |
| 11 | Delta LNGFR | atgggggcaggtgccaccggccgcgccatggacgggccgcgcctgctgctg |
| | | ttgctgcttctgggggtgtcccttggaggtgccaaggaggcatgccccacagg |
| | | cctgtacacacacagcggtgagtgctgcaaagcctgcaacctgggcgagggt |
| | | gtggcccagccttgtggagccaaccagaccgtgtgtgagccctgcctggacag |
| | | cgtgacgttctccgacgtggtgagcgcgaccgagccgtgcaagccgtgcacc |
| | | gagtgcgtggggctccagagcatgtcggcgccgtgcgtggaggccgacgac |
| | | gccgtgtgccgctgcgcctacggctactaccaggatgagacgactgggcgct |
| | | gcgaggcgtgccgcgtgtgcgaggcgggctcgggcctcgtgttctcctgcca |
| | | ggacaagcagaacaccgtgtgcgaggagtgccccgacggcacgtattccgac |
| | | gaggccaaccacgtggacccgtgcctgccctgcaccgtgtgcgaggacaccg |
| | | agcgccagctccgcgagtgcacacgctgggccgacgccgagtgcgaggag |
| | | atccctggccgttggattacacggtccacacccccagagggctcggacagcac |
| | | agcccccagcacccaggagcctgaggcacctccagaacaagacctcatagcc |
| | | agcacggtggcaggtgtggtgaccacagtgatgggcagctcccagcccgtgg |
| | | tgacccgaggcaccaccgacaacctcatccctgtctattgctccatcctggctgc |
| | | tgtggttgtgggtcttgtggcctacatagccttcaagaggtga |
| 12 | SV40 polyA | cagacatgataagatacattgatgagtttggacaaaccacaactagaatgcagt |
| | | gaaaaaaatgctttatttgtgaaatttgtgatgctattgctttatttgtaaccattataa |
| | | gctgcaataaacaagttaacaacaacaattgcattcattttatgtttcaggttcagg |
| | | gggaggtgtgggaggttttttaaagcaagtaaaacctctacaaatgtggta |
| 13 | left AAV2 ITR | cctgcaggcagctgcgcgctcgctcgctcactgaggccgcccgggcaaagcc |
| | | cgggcgtcgggcgacctttggtcgcccggcctcagtgagcgagcgagcgcg |
| | | cagagagggagtggccaactccatcactaggggttcct |
| 14 | right AAV2 ITR | aggaacccctagtgatggagttggccactccctctctgcgcgctcgctcgctca |
| | | ctgaggccgggcgaccaaaggtcgcccgacgcccgggctttgcccgggcgg |
| | | cctcagtgagcgagcgagcgcgcagctgcctgcagg |
| 15 | FOXP3.PKGprom.NGF.ITR | cctgcaggcagctgcgcgctcgctcgctcactgaggccgcccgggcaaagcc |
| | | cgggcgtcgggcgacctttggtcgcccggcctcagtgagcgagcgagcgcg |
| | | cagagagggagtggccaactccatcactaggggttcctgcggccgcagagga |
| | | ggaggccgtattgtagtcccatgagcatagctatgtgtccccatccccatgtgac |
| | | aagagaagaggactggggccaagtaggtgaggtgacagggctgaggccag |
| | | ctctgcaacttattagctgtttgatctttaaaaagttactcgatctccatgagcctca |
| | | gtttccatacgtgtaaaagggggatgatcatagcatctaccatgtgggcttgcag |
| | | tgcagagtatttgaattagacacagaacagtgaggatcaggatggcctctcacc |
| | | cacctgcctttctgcccagctgcccacactgcccctagtcatggtggcaccctcc |
| | | ggggcacggctgggccccttgccccacttacaggcactcctccaggacaggc |
| | | cacatttcatgcaccaggtatggacggtgaatgggcagggaggagggagcag |
| | | gtgggagaactgtggggaggggccccgagtcaggctgaaccacagcccaca |
| | | tgtgccccccagtaccacatttgtagaggttttacttgctttaaaaaacctcccaca |
| | | cctccccctgaacctgaaacataaaatgaatgcaattgttgttgttaacttgtttatt |
| | | gcagcttataatggttacaaataaagcaatagcatcacaaatttcacaaataaag |
| | | catttttttcactgcattctagttgtggtttgtccaaactcatcaatgtatottatcatgt |
| | | ctgctcgaagcggccggccgctatcacctcttgaaggctatgtaggccacaag |
| | | acccacaaccacagcagccaggatggagcaatagacagggatgaggttgtcg |
| | | gtggtgcctcgggtcaccacgggctgggagctgcccatcactgtggtcaccac |
| | | acctgccaccgtgctggctatgaggtcttgttctggaggtgcctcaggctcctgg |
| | | gtgctgggggctgtgctgtccgagccctctgggggtgtggaccgtgtaatcca |
| | | acggccagggatctcctcgcactcggcgtcggcccagcgtgtgcactcgcgg |
| | | agctggcgctcggtgtcctcgcacacggtgcagggcaggcacgggtccacgt |
| | | ggttggcctcgtcggaatacgtgccgtcggggcactcctcgcacacggtgttct |
| | | gcttgtcctggcaggagaacacgaggcccgagcccgcctcgcacacgcggc |
| | | acgcctcgcagcgcccagtcgtctcatcctggtagtagccgtaggcgcagcgg |
| | | cacacggcgtcgtcggcctccacgcacggcgccgacatgctctggagcccca |
| | | cgcactcggtgcacggcttgcacggctcggtcgcgctcaccacgtcggagaa |
| | | cgtcacgctgtccaggcagggctcacacacggtctggttggctccacaaggct |
| | | gggccacaccctcgcccaggttgcaggctttgcagcactcaccgctgtgtgtgt |
| | | acaggcctgtggggcatgcctccttggcacctccaagggacacccccagaag |
| | | cagcaacagcagcaggcgcggcccgtccatggcgcggccggtggcacctgc |
| | | ccccatggtggcgaattccgatctgacggttcactaaacgagctctgcttatata |
| | | ggcctcccaccgtacacgccacctcgacataaattctaccgggtaggggaggc |
| | | gcttttcccaaggcagtctggagcatgcgctttagcagccccgctgggcacttg |
| | | gcgctacacaagtggcctctggcctcgcacacattccacatccaccggtaggc |
| | | gccaaccggctccgttctttggtggccccttcgcgccaccttctactcctccccta |
| | | gtcaggaagttcccccccgccccgcagctcgcgtcgtgcaggacgtgacaaat |
| | | ggaagtagcacgtctcactagtctcgtgcagatggacagcaccgctgagcaat |
| | | ggaagcgggtaggcctttggggcagcggccaatagcagctttgctccttcgctt |
| | | tctgggctcagaggctgggaaggggtgggtccgggggcgggctcaggggc |
| | | gggctcaggggcggggcgggcgcccgaaggtcctccggaggcccggcatt |
| | | ctgcacgcttcaaaagcgcacgtctgccgcgctgttctcctcttcctcatctccgg |
| | | gcctttcgaccttagacgcacacactcatcgaaaaaaatttggattattagaaga |
| | | gagaggtctgcggcttccacaccgtacagcgtggtttttcttctcggtataaaagc |
| | | aaagttgtttttgatacgtgacagtttcccacaagccaggctgatccttttctgtca |
| | | gtccacttcaccaagcctgcccttggacaaggacccgatgcccaaccccaggc |
| | | ctggcaagccctcggccccttccttggcccttggcccatccccaggagcctcgc |
| | | ccagctggagggctgcacccaaagcctcagacctgctgggggcccggggcc |
| | | cagggggaaccttccagggccgagatcttcgaggcggggcccatgcctcctct |
| | | tcttccttgaaccccatgccaccatcgcagctgcagctgcccacactgccccta |
| | | gtcatggtggcaccctccggggcacggctgggccccttgccccacttacaggc |
| | | actcctccaggacaggccacatttcatgcaccagctctcaacggtggatgccca |
| | | cgcccggacccctgtgctgcaggtgcaccccctggagagcccagccatgatc |
| | | agcctcacaccacccaccaccgccactggggtcttctccctcaaggcccggcc |
| | | tggcctcccacctggtaacacctcagcccgtaccccatggcttcacagaacccc |
| | | caagtccccagatccttggctgtgagcagtgtaggctattctgaattgcagtactc |
| | | tgggggtcaaaggtgtcaggtctcagaggcttggaaactccaccctccaaaaa |
| | | acgtcaggtgcagaaccttaaagatgcagaatgtcaaaatcacaaaaccacag |
| | | agctttacaaagctagtcaaaatgtcagcacctgcgaatggccgtctttaagcttc |
| | | tctgccagaagcctgggactttggggacagcagagccccctgggagtcaggg |
| | | ttttcgaggctcaggagggtgggaagctcaaaatgagaggccttgtgggccgc |
| | | ggccgcaggaacccctagtgatggagttggccactccctctctgcgcgctcgct |
| | | cgctcactgaggccgggcgaccaaaggtcgcccgacgcccgggctttgccc |
| | | gggcggcctcagtgagcgagcgagcgcgcagctgcctgcaggggcgcctg |
| | | atgcggtattttctccttacgcatctgtgcggtatttcacaccgcatacgtcaaagc |
| | | aaccatagtacgcgccctgtagcggcgcattaagcgcggcgggtgtggtggtt |
| | | acgcgcagcgtgaccgctacacttgccagcgccctagcgcccgctcctttcgct |
| | | ttcttcccttcctttctcgccacgttcgccggctttccccgtcaagctctaaatcgg |
| | | gggctccctttagggttccgatttagtgctttacggcacctcgaccccaaaaaact |
| | | tgatttgggtgatggttcacgtagtgggccatcgccctgatagacggtttttcgcc |
| | | ctttgacgttggagtccacgttctttaatagtggactcttgttccaaactggaacaa |
| | | cactcaaccctatctcgggctattcttttgatttataagggattttgccgatttcggc |
| | | ctattggttaaaaaatgagctgatttaacaaaaatttaacgcgaattttaacaaaat |
| | | attaacgtttacaattttatggtgcactctcagtacaatctgctctgatgccgcatag |
| | | ttaagccagccccgacacccgccaacacccgctgacgcgccctgacgggctt |
| | | gtctgctcccggcatccgcttacagacaagctgtgaccgtctccgggagctgca |
| | | tgtgtcagaggttttcaccgtcatcaccgaaacgcgcgagacgaaagggcctc |
| | | gtgatacgcctatttttataggttaatgtcatgataataatggtttcttagacgtcag |
| | | gtggcacttttcggggaaatgtgcgcggaacccctatttgtttatttttctaaataca |
| | | ttcaaatatgtatccgctcatgagacaataaccctgataaatgcttcaataatattg |
| | | aaaaaggaagagtatgagtattcaacatttccgtgtcgcccttattcccttttttgcg |
| | | gcattttgccttcctgtttttgctcacccagaaacgctggtgaaagtaaaagatgct |
| | | gaagatcagttgggtgcacgagtgggttacatcgaactggatctcaacagcggt |
| | | aagatccttgagagttttcgccccgaagaacgttttccaatgatgagcacttttaa |
| | | agttctgctatgtggcgcggtattatcccgtattgacgccgggcaagagcaactc |
| | | ggtcgccgcatacactattctcagaatgacttggttgagtactcaccagtcacag |
| | | aaaagcatcttacggatggcatgacagtaagagaattatgcagtgctgccataa |
| | | ccatgagtgataacactgcggccaacttacttctgacaacgatcggaggaccga |
| | | aggagctaaccgcttttttgcacaacatgggggatcatgtaactcgccttgatcgt |
| | | tgggaaccggagctgaatgaagccataccaaacgacgagcgtgacaccacg |
| | | atgcctgtagcaatggcaacaacgttgcgcaaactattaactggcgaactactta |
| | | ctctagcttcccggcaacaattaatagactggatggaggcggataaagttgcag |
| | | gaccacttctgcgctcggcccttccggctggctggtttattgctgataaatctgga |
| | | gccggtgagcgtgggtctcgcggtatcattgcagcactggggccagatggtaa |
| | | gccctcccgtatcgtagttatctacacgacggggagtcaggcaactatggatga |
| | | acgaaatagacagatcgctgagataggtgcctcactgattaagcattggtaactg |
| | | tcagaccaagtttactcatatatactttagattgatttaaaacttcatttttaatttaaaa |
| | | ggatctaggtgaagatcctttttgataatctcatgaccaaaatcccttaacgtgagt |
| | | tttcgttccactgagcgtcagaccccgtagaaaagatcaaaggatcttcttgagat |
| | | cctttttttctgcgcgtaatctgctgcttgcaaacaaaaaaaccaccgctaccagc |
| | | ggtggtttgtttgccggatcaagagctaccaactctttttccgaaggtaactggctt |
| | | cagcagagcgcagataccaaatactgtccttctagtgtagccgtagttaggcca |
| | | ccacttcaagaactctgtagcaccgcctacatacctcgctctgctaatcctgttac |
| | | cagtggctgctgccagtggcgataagtcgtgtcttaccgggttggactcaagac |
| | | gatagttaccggataaggcgcagcggtcgggctgaacggggggttcgtgcac |
| | | acagcccagcttggagcgaacgacctacaccgaactgagatacctacagcgt |
| | | gagctatgagaaagcgccacgcttcccgaagggagaaag |
| 16 | FOXP3.UBCprom.NGFR.ITR | cctgcaggcagctgcgcgctcgctcgctcactgaggccgcccgggcaaagcc |
| | | cgggcgtcgggcgacctttggtcgcccggcctcagtgagcgagcgagcgcg |
| | | cagagagggagtggccaactccatcactaggggttcctgcggccgcagagga |
| | | ggaggccgtattgtagtcccatgagcatagctatgtgtccccatccccatgtgac |
| | | aagagaagaggactggggccaagtaggtgaggtgacagggctgaggccag |
| | | ctctgcaacttattagctgtttgatctttaaaaagttactcgatctccatgagcctca |
| | | gtttccatacgtgtaaaagggggatgatcatagcatctaccatgtgggcttgcag |
| | | tgcagagtatttgaattagacacagaacagtgaggatcaggatggcctctcacc |
| | | cacctgcctttctgcccagctgcccacactgcccctagtcatggtggcaccctcc |
| | | ggggcacggctgggccccttgccccacttacaggcactcctccaggacaggc |
| | | cacatttcatgcaccaggtatggacggtgaatgggcagggaggagggagcag |
| | | gtgggagaactgtggggaggggccccgagtcaggctgaaccacagcccaca |
| | | tgtgccccccagtaccacatttgtagaggttttacttgctttaaaaaacctcccaca |
| | | cctccccctgaacctgaaacataaaatgaatgcaattgttgttgttaacttgtttatt |
| | | gcagcttataatggttacaaataaagcaatagcatcacaaatttcacaaataaag |
| | | catttttttcactgcattctagttgtggtttgtccaaactcatcaatgtatcttatcatgt |
| | | ctgctcgaagcggccggccgctatcacctcttgaaggctatgtaggccacaag |
| | | acccacaaccacagcagccaggatggagcaatagacagggatgaggttgtcg |
| | | gtggtgcctcgggtcaccacgggctgggagctgcccatcactgtggtcaccac |
| | | acctgccaccgtgctggctatgaggtcttgttctggaggtgcctcaggctcctgg |
| | | gtgctgggggctgtgctgtccgagccctctgggggtgtggaccgtgtaatcca |
| | | acggccagggatctcctcgcactcggcgtcggcccagcgtgtgcactcgcgg |
| | | agctggcgctcggtgtcctcgcacacggtgcagggcaggcacgggtccacgt |
| | | ggttggcctcgtcggaatacgtgccgtcggggcactcctcgcacacggtgttct |
| | | gcttgtcctggcaggagaacacgaggcccgagcccgcctcgcacacgcggc |
| | | acgcctcgcagcgcccagtcgtctcatcctggtagtagccgtaggcgcagcgg |
| | | cacacggcgtcgtcggcctccacgcacggcgccgacatgctctggagcccca |
| | | cgcactcggtgcacggcttgcacggctcggtcgcgctcaccacgtcggagaa |
| | | cgtcacgctgtccaggcagggctcacacacggtctggttggctccacaaggct |
| | | gggccacaccctcgcccaggttgcaggctttgcagcactcaccgctgtgtgtgt |
| | | acaggcctgtggggcatgcctccttggcacctccaagggacacccccagaag |
| | | cagcaacagcagcaggcgcggcccgtccatggcgcggccggtggcacctgc |
| | | ccccatggtggcgaattccgatctgacggttcactaaacgagctctgcttatata |
| | | ggcctcccaccgtacacgccacctcgacataccgcggctaaggaacgcgggc |
| | | cgcccacccgctccgggtgcagcggcctccgcgccgggttttggcgcctccc |
| | | gcgggcgcccccctcctcacggcgagcgctgccacgtcagacgaagggcgc |
| | | agcgagcgtcctgatccttccgcccggacgctcaggacagcggcccgctgct |
| | | cataagactcggccttagaaccccagtatcagcagaaggacattttaggacgg |
| | | gacttgggtgactctagggcactggttttctttccagagagcggaacaggcgag |
| | | gaaaagtagtcccttctcggcgattctgcggagggatctccgtggggcggtga |
| | | acgccgatgattatataaggacgcgccgggtgtggcacagctagttccgtcgc |
| | | agccgggatttgggtcgcagttcttgtttgtggatcgctgtgatcgtcacttagtg |
| | | agtagcgggctgctgggctggccggggctttcgtggccgccgggccgctcgg |
| | | tgggacggaagcgtgtggagagaccgccaagggctgtagtctgggtccgcg |
| | | agcaaggttgccctgaactgggggttggggggagcgcagcaaaatggcggc |
| | | tgttcccgagtcttgaatggaagacgcttgtgaggcgggctgtgaggtcgttga |
| | | aacaaggtggagggcatggtgggcggcaagaacccaaggtcttgaggccttc |
| | | gctaatgcgggaaagctcttattcgggtgagatgggctggggcaccatctggg |
| | | gaccctgacgtgaagtttgtcactgactggagaactcggtttgtcgtctgttgcg |
| | | ggggcggcagttatggcggtgccgttgggcagtgcacccgtacctttgggagc |
| | | gcgcgccctcgtcgtgtcgtgacgtcacccgttctgttggcttataatgcagggt |
| | | ggggccacctgccggtaggtgtgcggtaggcttttctccgtcgcaggacgcag |
| | | ggttcgggcctagggtaggctctcctgaatcgacaggcgccggacctctggtg |
| | | ggggagggataagtgaggcgtcagtttctttggtcggttttatgtacctatcttct |
| | | taagtagctgaagctccggttttgaactatgcgctcggggttggcgagtgtgtttt |
| | | gtgaagttttttaggcaccttttgaaatgtaatcatttgggtcaatatgtaattttcagt |
| | | gttagactagtaaattgtccgctaaattctggccgtttttggcttttttgttagacgca |
| | | cacactcatcgaaaaaaatttggattattagaagagagaggtctgcggcttccac |
| | | accgtacagcgtggtttttcttctcggtataaaagcaaagttgtttttgatacgtgac |
| | | agtttcccacaagccaggctgatccttttctgtcagtccacttcaccaagcctgcc |
| | | cttggacaaggacccgatgcccaaccccaggcctggcaagccctcggcccct |
| | | tccttggcccttggcccatccccaggagcctcgcccagctggagggctgcacc |
| | | caaagcctcagacctgctgggggcccggggcccagggggaaccttccaggg |
| | | ccgagatcttcgaggggggcccatgcctcctcttcttccttgaaccccatgcca |
| | | ccatcgcagctgcagctgcccacactgcccctagtcatggtggcaccctccgg |
| | | ggcacggctgggccccttgccccacttacaggcactcctccaggacaggcca |
| | | catttcatgcaccagctctcaacggtggatgcccacgcccggacccctgtgctg |
| | | caggtgcaccccctggagagcccagccatgatcagcctcacaccacccacca |
| | | ccgccactggggtcttctccctcaaggcccggcctggcctcccacctggtaaca |
| | | cctcagcccgtaccccatggcttcacagaacccccaagtccccagatccttggc |
| | | tgtgagcagtgtaggctattctgaattgcagtactctgggggtcaaaggtgtcag |
| | | gtctcagaggcttggaaactccaccctccaaaaaacgtcaggtgcagaacctta |
| | | aagatgcagaatgtcaaaatcacaaaaccacagagctttacaaagctagtcaaa |
| | | atgtcagcacctgcgaatggccgtctttaagcttctctgccagaagcctgggact |
| | | ttggggacagcagagccccctgggagtcagggttttcgaggctcaggagggt |
| | | gggaagctcaaaatgagaggccttgtgggccgcggccgcaggaacccctagt |
| | | gatggagttggccactccctctctgcgcgctcgctcgctcactgaggccgggc |
| | | gaccaaaggtcgcccgacgcccgggctttgcccgggcggcctcagtgagcg |
| | | agcgagcgcgcagctgcctgcaggggcgcctgatgcggtattttctccttacgc |
| | | atctgtgcggtatttcacaccgcatacgtcaaagcaaccatagtacgcgccctgt |
| | | agcggcgcattaagcgcggcgggtgtggtggttacgcgcagcgtgaccgcta |
| | | cacttgccagcgccctagcgcccgctcctttcgctttcttcccttcctttctcgcca |
| | | cgttcgccggctttccccgtcaagctctaaatcgggggctccctttagggttccg |
| | | atttagtgctttacggcacctcgaccccaaaaaacttgatttgggtgatggttcac |
| | | gtagtgggccatcgccctgatagacggtttttcgccctttgacgttggagtccac |
| | | gttctttaatagtggactcttgttccaaactggaacaacactcaaccctatctcggg |
| | | ctattcttttgatttataagggattttgccgatttcggcctattggttaaaaaatgagc |
| | | tgatttaacaaaaatttaacgcgaattttaacaaaatattaacgtttacaattttatgg |
| | | tgcactctcagtacaatctgctctgatgccgcatagttaagccagccccgacacc |
| | | cgccaacacccgctgacgcgccctgacgggcttgtctgctcccggcatccgct |
| | | tacagacaagctgtgaccgtctccgggagctgcatgtgtcagaggttttcaccgt |
| | | catcaccgaaacgcgcgagacgaaagggcctcgtgatacgcctatttttatagg |
| | | ttaatgtcatgataataatggtttcttagacgtcaggtggcacttttcggggaaatg |
| | | tgcgcggaacccctatttgtttatttttctaaatacattcaaatatgtatccgctcatg |
| | | agacaataaccctgataaatgcttcaataatattgaaaaaggaagagtatgagta |
| | | ttcaacatttccgtgtcgcccttattcccttttttgcggcattttgccttcctgtttttgc |
| | | tcacccagaaacgctggtgaaagtaaaagatgctgaagatcagttgggtgcac |
| | | gagtgggttacatcgaactggatctcaacagcggtaagatccttgagagttttcg |
| | | ccccgaagaacgttttccaatgatgagcacttttaaagttctgctatgtggcgcgg |
| | | tattatcccgtattgacgccgggcaagagcaactcggtcgccgcatacactattc |
| | | tcagaatgacttggttgagtactcaccagtcacagaaaagcatcttacggatggc |
| | | atgacagtaagagaattatgcagtgctgccataaccatgagtgataacactgcg |
| | | gccaacttacttctgacaacgatcggaggaccgaaggagctaaccgcttttttgc |
| | | acaacatgggggatcatgtaactcgccttgatcgttgggaaccggagctgaatg |
| | | aagccataccaaacgacgagcgtgacaccacgatgcctgtagcaatggcaac |
| | | aacgttgcgcaaactattaactggcgaactacttactctagcttcccggcaacaat |
| | | taatagactggatggaggcggataaagttgcaggaccacttctgcgctcggcc |
| | | cttccggctggctggtttattgctgataaatctggagccggtgagcgtgggtctc |
| | | gcggtatcattgcagcactggggccagatggtaagccccccgtatcgtagttat |
| | | ctacacgacggggagtcaggcaactatggatgaacgaaatagacagatcgct |
| | | gagataggtgcctcactgattaagcattggtaactgtcagaccaagtttactcata |
| | | tatactttagattgatttaaaacttcatttttaatttaaaaggatctaggtgaagatcct |
| | | ttttgataatctcatgaccaaaatcccttaacgtgagttttcgttccactgagcgtca |
| | | gaccccgtagaaaagatcaaaggatcttcttgagatcctttttttctgcgcgtaatc |
| | | tgctgcttgcaaacaaaaaaaccaccgctaccagcggtggtttgtttgccggatc |
| | | aagagctaccaactctttttccgaaggtaactggcttcagcagagcgcagatac |
| | | caaatactgtccttctagtgtagccgtagttaggccaccacttcaagaactctgta |
| | | gcaccgcctacatacctcgctctgctaatcctgttaccagtggctgctgccagtg |
| | | gcgataagtcgtgtcttaccgggttggactcaagacgatagttaccggataagg |
| | | cgcagcggtcgggctgaacggggggttcgtgcacacagcccagcttggagc |
| | | gaacgacctacaccgaactgagatacctacagcgtgagctatgagaaagcgc |
| | | cacgcttcccgaagggagaaaggcggacaggtatccggtaagcggcagggt |
| | | cggaacaggagagcgcacgagggagcttccagggggaaacgcctggtatctt |
| | | tatagtcctgtcgggtttcgccacctctgacttgagcgtcgatttttgtgatgctcgt |
| | | caggggggcggagcctatggaaaaacgccagcaacgcggcctttttacggttc |
| | | ctggccttttgctggccttttgctcacatgt |
| 17 | consensus sequence | RXK(R)R |

### Example 2—General Procedure for CD4+ T Cell Engineering

Human PBMCs, obtained by apheresis of healthy donors and viable CD4+ T cells were purified from PBMCs by negative selection using the EasySep Human CD4+ T Cell Enrichment Kit (STEMCELL Technologies) and then either frozen for later use or directly cultured for editing.

CD4+ T cells were thawed and 1×10⁶ cells/ml were activated in T cell media using anti-CD3/anti-CD28 Dynabeads at a bead:cell ratio of 1:1 in T cell media. CD4+ T cells in the absence of anti-CD3/anti-CD28 Dynabeads were used as unstimulated control cells.

AAV6.FOXP3.NGFR donor vectors were thawed and 1×10³ gene copies of viral vector/cell were added to the CD4+ T cell/Dynabead cultures.

TracrRNA:crRNA complexes were annealed by combining at a 1:1 ratio of 20 μM tracrRNA and 20 μM crRNA (1001M each final concentration) and incubated at 95° C. for 5 min. followed by a cool down period of about 10 min at room temperature. The crRNA target sequence used was: ATCCACCGTTGAGAGCTGGG (SEQ ID NO: 1).

The ribonucleoprotein (RNP) complex was generated by mixing 17 μg Cas9, 120pmol annealed RNA complex, and 100pmol ssODN (enhances electroporation of RNP complex) and incubating the mixture for 10 min. at room temperature.

The anti-CD3/anti-CD28 Dynabead stimulated CD4+ T cells were washed several times and resuspended in complete P3 Nucleofector solution. The preformed RNP complexes were added to the T cell-Nucleofector solution and nucleofection was induced using program CM-138 on the Amaxa 4D-Nucleofector for stimulated cells or program DS-137 on the Amaxa 4D-Nucleofector for non-stimulated cells.

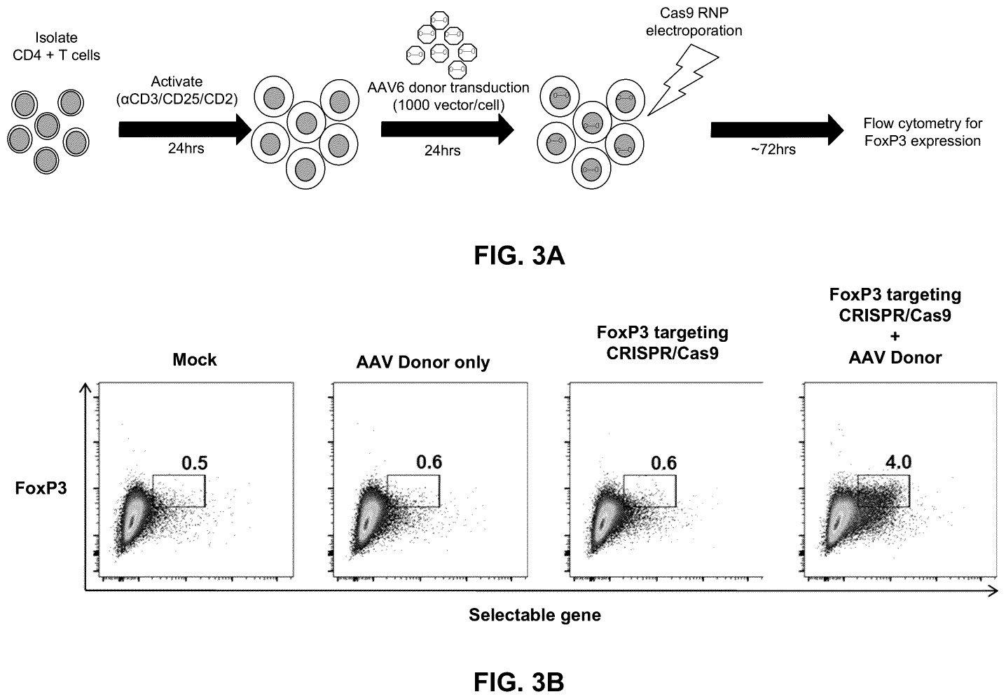

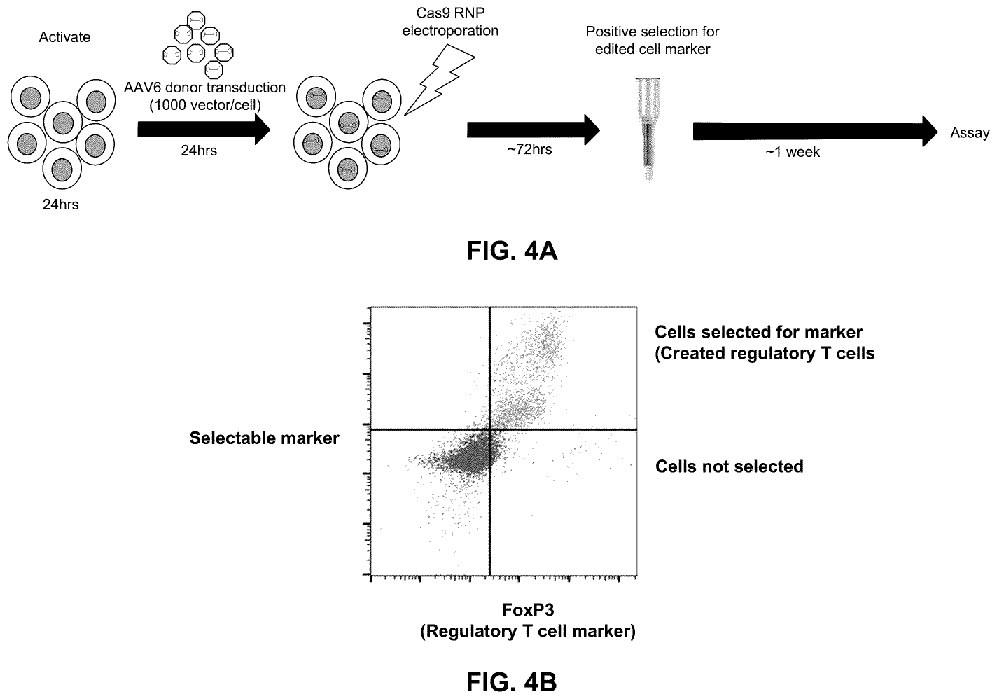

The FOXP3 engineered T cells were subsequently washed in StemCell RoboSep Buffer II and isolated using MACs separation (Miltenyi kit 130-099-023). To this end, about 10⁷ FOXP3 engineered T cells in 60 μl StemCell RoboSep Buffer were combined with 20 μl of Miltenyi FcR blocking reagent and 20 μl of Miltenyi CD271 microbeads blocking reagent, the mixture was incubated for 15 min. at 4° C. and the cells were separated on a LS MACs column. The experimental procedure is shown in FIG. 3A. Engineered T cells were either stored for further processing or submitted to FACS analysis for the expression of FOXP3 and truncated NGFR. Flow cytometric analysis of engineered T cells revealed a population of T cells expressing both, FOXP3 and truncated NGFR (FIG. 3B). Further culture of NGFR-selected, engineered T cells for 1 week allowed the purification of a population of FOXP3-engineered cells (FIGS. 4A and 4B).

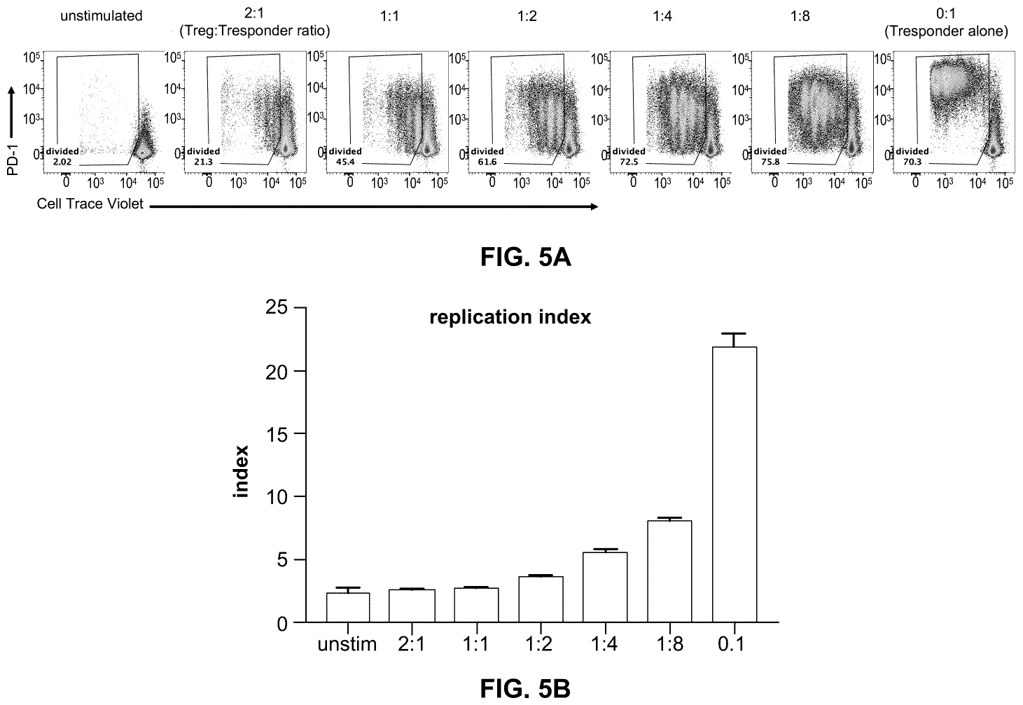

Engineered, purified FOXP3 T cells were tested for suppressor function by co-culturing the engineered, purified FOXP3 T cells with CD25−CD4+ T cells (“responder T cells”) for 4 days after which time the responder T cells were assayed for proliferation as demonstrated by dilution of Cell Trace Violet. The responder T cells were also phenotyped for several surface markers that are indicators for T cell activation. Furthermore, the supernatant of responder T cell cultures was collected and assayed for cytokines. FIG. 5A shows flow cytometry scans of cell trace violet (x-axis) and PD-1 expression (y-axis) of unstimulated responder T cells or responder T cells stimulated with a T cell stimulation reagent (anti-CD3, anti-CD28, anti-CD2) in the absence of engineered, purified FOXP3 Tregs or co-cultured with the engineered, purified FOXP3 Treg cells at different Treg to T responder cell ratios. Unstimulated responder T cells did not proliferate (FIG. 5A left panel, 2.02% proliferating T cells), while 70.3% of stimulated responder T cells proliferated in the absence of engineered Treg (FIG. 5A panel on the right). With increasing Treg to responder T cell ratios, the percentage of proliferating responder T cells was reduced (FIG. 5A, first to sixth panels). FIG. 5B shows a histogram of replication indices of unstimulated and stimulated responder T cells following co-cultures with engineered Tregs at different Treg to T responder cell ratios. These data demonstrated that engineered, purified Treg cells efficiently inhibited the proliferation of stimulated responder T cells.

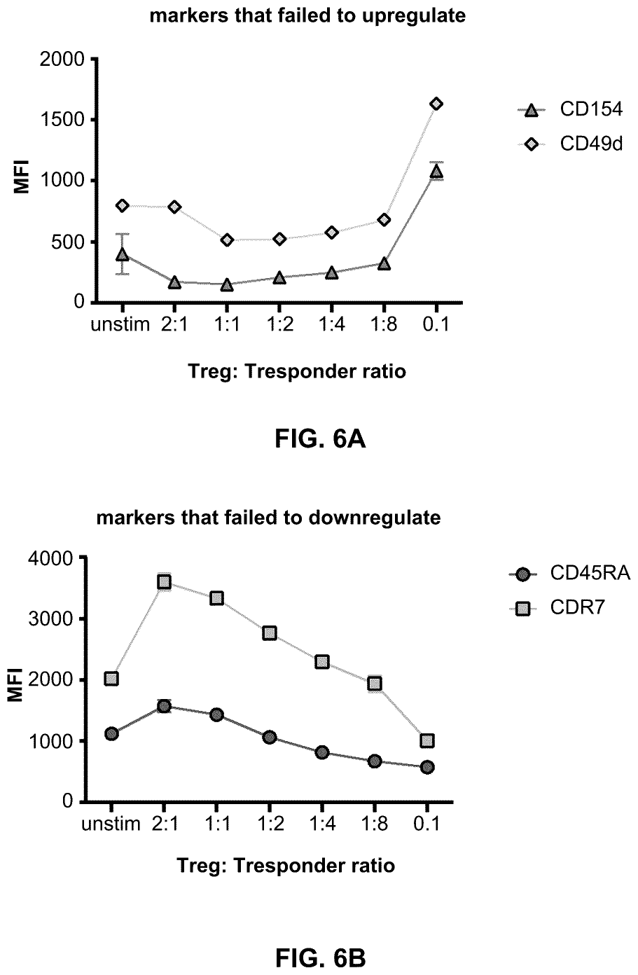

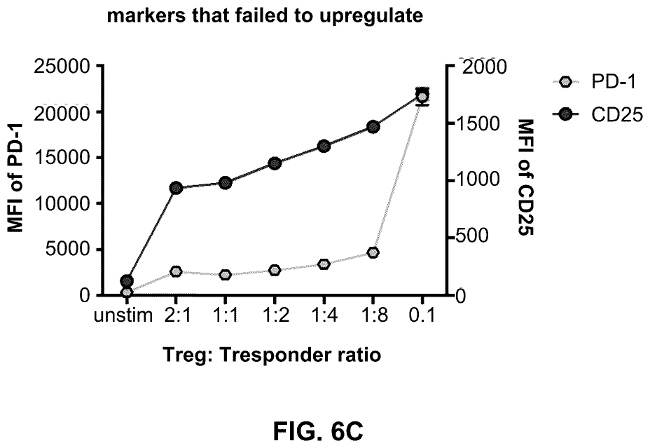

Cell surface markers CD154, CD49d, PD-1, CD25, CD45RA and CCR7 were detected on unstimulated responder T cells and responder T cells stimulated with a T cell stimulation reagent (anti-CD3, anti-CD28, anti-CD2) in the absence or presence of engineered FOXP3 T cells at different Treg: responder T cell ratios. It could readily be observed that engineered FOXP3 Tregs prevented the upregulation of CD154, CD49d, PD-1 and CD25 on responder T cells (FIGS. 6A and 6B). Furthermore, engineered FOXP3 Treg cells prevented the downregulation of CD45RA and CCR7 on responder T cells (FIG. 6C).

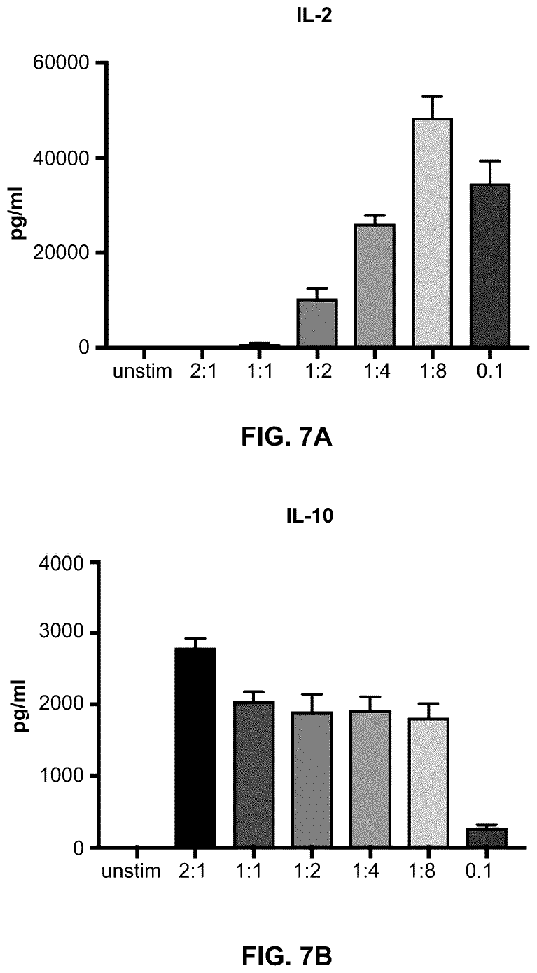

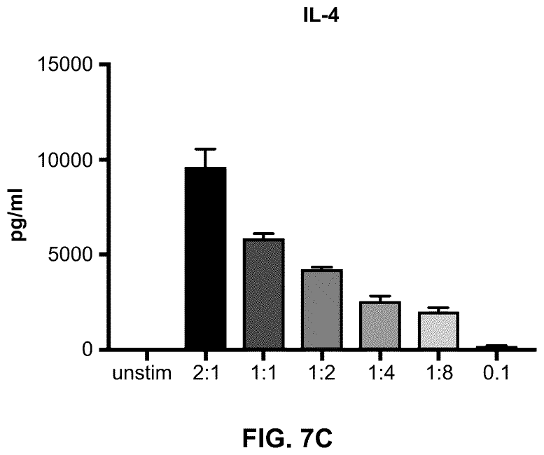

Secreted cytokines IL-2, IL-4 and IL-10 were measured in supernatants of responder T cells cultures in unstimulated responder T cells and responder T cells stimulated with a T cell stimulation reagent (anti-CD3, anti-CD28, anti-CD2) in the absence or presence of engineered FOXP3 T cells at different Treg: responder T cell ratios. It could readily be observed that engineered FOXP3 Tregs suppressed IL-2 secretion FIG. 7A) and increased secretion of anti-inflammatory cytokines IL-10 and IL-4 (FIGS. 7B and 7C).

### Example 3—Generation of Engineered FOXP3 T Cells that Specifically Suppress Responder T Cell Activation by Porcine Cells

Human PBMCs, obtained by apheresis of healthy donors and viable CD4+ T cells were purified from PBMCs by negative selection using the EasySep Human CD4+ T Cell Enrichment Kit (STEMCELL Technologies) and then either frozen for later use or directly cultured for editing.

CD4+ T cells were thawed and 1×10⁶ cells/ml were activated in T cell media using anti-CD3/anti-CD28 Dynabeads at a bead:cell ratio of 1:1 in T cell media. CD4+T cells in the absence of anti-CD3/anti-CD28 Dynabeads were used as unstimulated control cells.

AAV6.FOXP3.NGFR donor vectors were thawed and 1×10³ gene copies of viral vector/cell were added to the CD4+ T cell/Dynabead cultures.

TracrRNA:crRNA complexes were annealed by combining at a 1:1 ratio of 20 μM tracrRNA and 20 μM crRNA (100M each final concentration) and incubated at 95° C. for 5 min. followed by a cool down period of about 10 min at room temperature. The crRNA target sequence was: ATCCACCGTTGAGAGCTGGG.

The ribonucleoprotein (RNP) complex was generated by mixing 17 μg Cas9, 120pmol annealed RNA complex, and 100pmol ssODN (enhances electroporation of RNP complex) and incubating the mixture for 10 min. at room temperature.

The anti-CD3/anti-CD28 Dynabead stimulated CD4+ T cells were washed several times and resuspended in complete P3 Nucleofector solution. The preformed RNP complexes were added to the T cell-Nucleofector solution and nucleofection was induced using program CM-138 on the Amaxa 4D-Nucleofector for stimulated cells or program DS-137 on the Amaxa 4D-Nucleofector for non-stimulated cells.

The FOXP3 engineered T cells were subsequently washed in StemCell RoboSep Buffer II and isolated using MACs separation (Miltenyi kit 130-099-023). To this end, about 10⁷ FOXP3 engineered T cells in 60 μl StemCell RoboSep Buffer were combined with 20 μl of Miltenyi FcR blocking reagent and 20 μl of Miltenyi CD271 microbeads blocking reagent, the mixture was incubated for 15 min. at 4° C. and the cells were separated on a LS MACs column. The engineered FOXP3 T cells retrieved from the LS MACs column were suspended in T cell medium with 400U/ml IL-2.

Fresh CD4+ cells from the same donor as the CD4+ cells used for generating engineered FOXP3 Treg cells were used as responder T cells. The fresh CD4+T responder cells were stained with Cell Trace Violet and 1×10⁶ fresh CD4+T responder cells were mixed with engineered FOXP3 Treg cells at the ratios of 1:1, 1:2, 1:4, 1:8, and 1:16 Treg to fresh CD4+T responder cells. An equivalent of 1×10⁴fresh CD4+T responder cells were incubated with 1×10⁴ primary porcine Pulmonary Artery Endothelial cells. The cell mixture was incubated at 37° C.+5% CO₂ in a culture dish for 7 days and responder T cells were subsequently assayed for proliferation as demonstrated by dilution of Cell Trace Violet. The responder T cells were also phenotyped for activation surface markers.

To discern between responder T cells and engineered FOXP3 Treg cells, the cells were stained for NGFR before phenotyping such that NGFR-expressing engineered FOXP3 Tregs could be excluded from the phenotyping analysis.

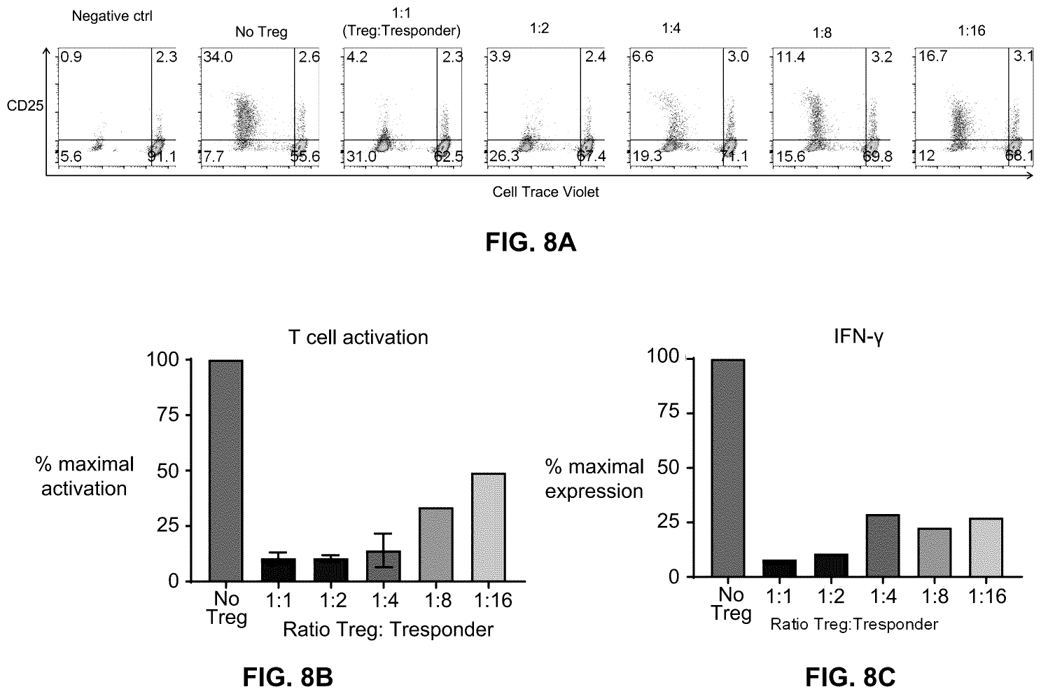

The flow cytometry scan of Cell Trace Violet dilution and CD25 expression of unstimulated responder T cells showed low proliferation, while the scan of stimulated responder T cells in the absence of engineered T reg cells showed high cell proliferation (FIG. 8A left two panels). In the presence of engineered Treg cells at increasing Treg: responder T cell ratios, the proliferation of responder T cells was reduced (FIG. 8A, compare seventh to third panel). The percentage of maximal activation of stimulated responder T cells in the absence of engineered Treg cells was suppressed with increasing Treg:responder T cell ratios (FIG. 8B) and the percentage of maximal IFN-7 secretion was also suppressed with increasing Treg:responder T cell ratios (FIG. 8C).

These results demonstrate that engineered FOXP3 Treg cells can efficiently suppress the activation of responder T cells that are stimulated by porcine cells. These results have implications for the use of engineered FOXP3 Treg cells in xenotransplantation.

### Example 5—Generation of Engineered FOXP3 T Cells that Specifically Suppress Responder T Cell Activation by Unmatched Human Donor Cells

Human PBMCs, obtained by apheresis of healthy donors and viable CD4+ T cells were purified from PBMCs by negative selection using the EasySep Human CD4+ T Cell Enrichment Kit (STEMCELL Technologies) and then either frozen for later use or directly cultured for editing.

Fresh CD4+ cells were activated on unmatched primary human Pulmonary Artery Endothelial Cells by incubating 1×10⁴ CD4+ T cells with 1×10⁴ unmatched primary human Pulmonary Artery Endothelial Cells (allo-hPAEC). The cell mixture was cultured for 2 days at 37° C.+5% CO2. As controls, non-activated fresh CD4+ cells from the same donor were used to generate bulk Treg cells. On the second day of activation of CD4+ T cells on allo-hPAECs, the non-adherent CD4+ T cells were removed from the adherent allo-hPAECs. The now allo-hPAEC-stimulated CD4+ T cells were washed and 1×10⁶ T cells/ml T cell medium of allo-hPAEC-stimulated and unstimulated T cells were incubated with 50 U/ml IL-2 and further cultured for 1 day at 37° C.+5% CO₂.

AAV6.FOXP3.NGFR donor vectors were thawed and 1×10³ gene copies of viral vector/cell were added to the CD4+ T cell/Dynabead cultures.

TracrRNA:crRNA complexes were annealed by combining at a 1:1 ratio of 20 μM tracrRNA and 20 μM crRNA (100M each final concentration) and incubated at 95° C. for 5 min. followed by a cool down period of about 10 min at room temperature. The crRNA target sequence was: ATCCACCGTTGAGAGCTGGG (SEQ ID NO: 1).

The ribonucleoprotein (RNP) complex was generated by mixing 17 g Cas9, 120pmol annealed RNA complex, and 100pmol ssODN (enhances electroporation of RNP complex) and incubating the mixture for 10 min. at room temperature.

The allo-hPAEC-stimulated and unstimulated T cells were washed several times and resuspended in complete P3 Nucleofector solution. The preformed RNP complexes were added to the T cell-Nucleofector solutions and nucleofection was induced using program CM-138 on the Amaxa 4D-Nucleofector for allo-hPAEC-stimulated cells. The unstimulated T control cells were nucleofected using program DS-137 on the Amaxa 4D-Nucleofector.

The FOXP3 engineered, allo-hPAEC-stimulated T cells and FOXP3 engineered, unstimulated T cells were subsequently incubated at room temperature for 5-10 minutes and transferred to T cell medium for a 1-day culture. The FOXP3 engineered, allo-hPAEC-stimulated and unstimulated T cell were then washed in StemCell RoboSep Buffer II and isolated using MACs separation (Miltenyi kit 130-099-023) as described above. The isolated FOXP3 engineered, allo-hPAEC-stimulated and unstimulated T cells were incubated in 100 μl T cell medium and 400 Units/ml of IL-2.

Fresh CD4+ T cells from the same donor as the engineered, allo-hPAEC-stimulated and engineered, unstimulated Treg cells were used as responder T cells.

Fresh CD4+ responder T cells were mixed with the FOXP3 engineered, allo-hPAEC-stimulated and unstimulated T cells at different Treg:responder T cell ratios of 1:1, 1:2, 1:4, 1:8, 1:16, and 1:32 Treg:responder T cells. Subsequently, an equivalent of 1×10⁴ fresh CD4+T responder cells were mixed with 1×10⁴ allo-hPAEC-stimulated and unstimulated Treg cells and the cell mixtures were incubated at 37° C.+5% CO₂ in a culture dish for 7 days. The responder T cells were subsequently assayed for proliferation as demonstrated by dilution of Cell Trace Violet and were phenotyped for activation surface markers.

To discern between responder T cells and engineered FOXP3 Treg cells, the cells were stained for NGFR before phenotyping such that NGFR-expressing engineered FOXP3 Tregs could be excluded from the phenotyping analysis.

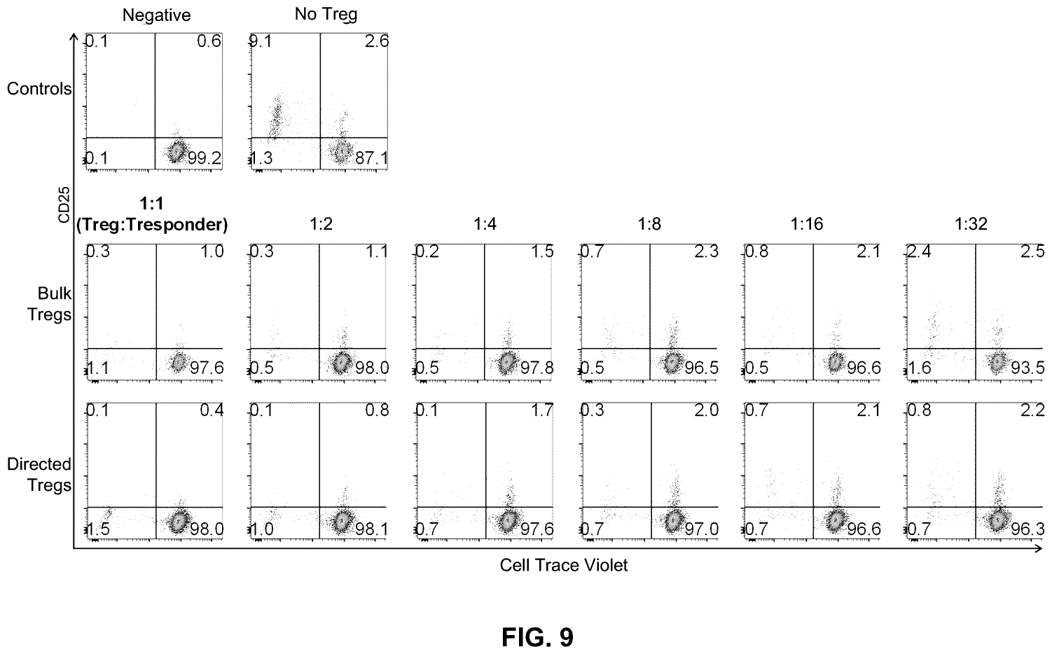

The flow cytometry scan of Cell Trace Violet dilution and CD25 expression of unstimulated responder T cells showed low proliferation, while the scan of stimulated responder T cells in the absence of engineered Treg cells showed high cell proliferation (FIG. 9 two panels on the top). In the presence of bulk engineered Treg cells and Tregs directed to target cells (directed Tregs) at increasing Treg:responder T cell ratios, the proliferation of responder T cells was suppressed (FIG. 9, second and third row panels).

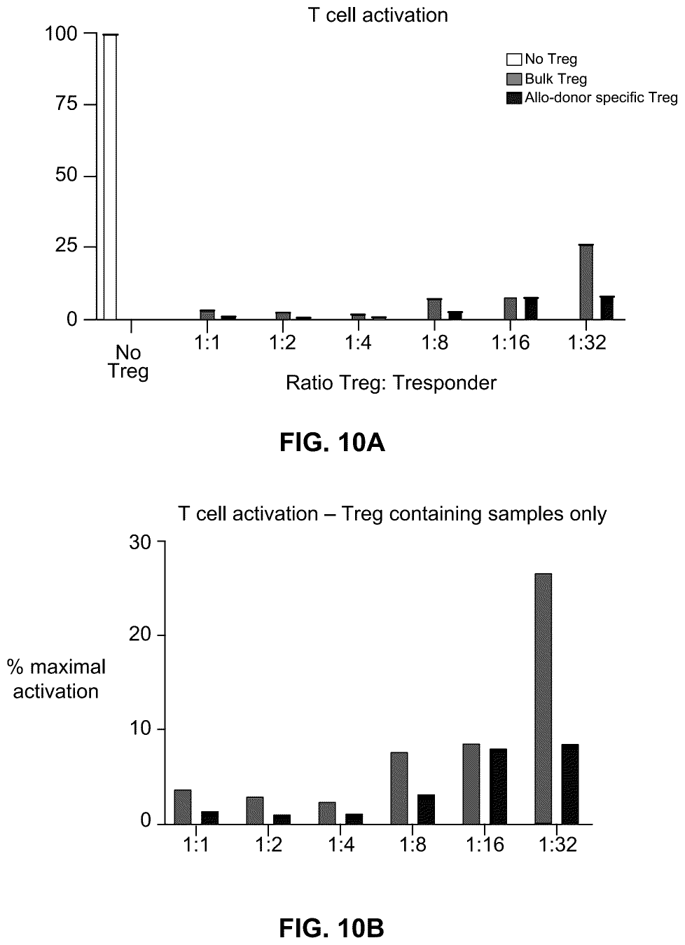

The T cell activation of stimulated responder T cells in the absence of engineered Treg cells and in the presence of bulk Tregs or allo-hPAEC-specific Tregs showed efficient suppression of responder T cell activation at all Treg:responder T cell ratios (FIG. 10A).

Further, the percentage of maximal T cell activation of responder T cells in the presence of bulk Tregs or allo-hPAEC-specific Tregs also demonstrated effective suppression of responder T cell activation with both Treg populations. Only at a Treg:responder T cell ratio of 1:32 were bulk Treg cells not as efficient at suppressing responder T cell activation as allo-hPAEC-specific Tregs (FIG. 10B).

These results demonstrate that engineered, human Pulmonary Artery Endothelial Cell-specific FOXP3 Treg cells and engineered FOXP3 bulk Tregs can efficiently suppress the activation of responder T cells by human Pulmonary Artery Endothelial Cells. These results have implications for the use of engineered FOXP3 Treg cells in cell and/or tissue transplantation.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

1. A method of making a polynucleotide for expression of FOXP3, the method comprising: (i) providing a first nucleotide sequence, wherein the first nucleotide sequence comprises a coding strand and a targeted locus, the coding strand comprising one or more regulatory elements and a FOXP3 gene, and the targeted locus comprising an intron sequence of the FOXP3 gene; (ii) providing a second nucleic acid sequence that comprises a heterologous promoter operably linked to a polynucleotide comprising at least one FOXP3 exon or a portion of a FOXP3 exon; (iii) providing a nuclease; and (iv) performing a gene editing process on the first nucleotide sequence, which edits the intron sequence, and inserts the second nucleic acid into the targeted locus, wherein insertion of the second nucleic acid results in expression of FOXP3.

2. The method of claim 1, wherein the second nucleotide sequence further comprises a polynucleotide encoding a selectable marker protein, wherein the selectable marker protein is a truncated, low-affinity nerve growth factor receptor protein.

3. The method of claim 1, wherein the promoter is a bidirectional promoter and controls transcription of the polynucleotide comprising the at least one FOXP3 exon or portion thereof and the polynucleotide encoding the selectable marker protein in opposite directions.

4-5. (canceled)

6. The method of claim 1, wherein i) the second nucleotide sequence comprises exons 1, 2, and 3 of FOXP3 and the targeted locus is at an intron between exon 2 and exon 3 of the FOXP3 gene of the first nucleotide sequence, or (ii) the second nucleotide sequence comprises exons 1, 2, 3, and 4 of FOXP3 and the targeted locus is at an intron between exon 3 and exon 4 of the FOXP3 gene of the first nucleotide sequence.

7-9. (canceled)

10. A polynucleotide comprising: a coding strand that comprises a heterologous promoter operably linked to a polynucleotide encoding a selectable marker protein and a first exon of a FOXP3 gene, wherein the polynucleotide further comprises a second and, optionally, a third exon of the FOXP3 gene and the heterologous promoter is located between the polynucleotide encoding the selectable marker protein and the first exon of the FOXP3 gene, wherein the heterologous promoter is bidirectional and promotes transcription of the polynucleotide encoding the selectable marker protein in one direction and the first exon of the FOXP3 gene in the opposite direction.

11-13. (canceled)

14. A system comprising a (i) polynucleotide of claim 10, and (ii) a nuclease.

15. The system of claim 14, wherein the polynucleotide further comprises a 5′ arm and a 3′ arm that each are homologous to a portion of an intron sequence of a FOXP3 gene.

16. The system of claim 15, wherein (i) the 5′ arm and the 3′ arm of the polynucleotide are each homologous to a portion of an intron that is located between a second and a third exon of a FOXP3 gene and wherein the polynucleotide comprises a first and a second FOXP3 exon or (ii) the 5′ arm and the 3′ arm of the polynucleotide are each homologous to a portion of an intron that is located between a third and a fourth exon of the FOXP3 gene and wherein the polynucleotide comprises a first, second, and third FOXP3 exon.

17-22. (canceled)

23. A method of suppressing T cell activation using an engineered regulatory T cell, the method comprising:
(i) inducing FOXP3 expression in a T cell using the polynucleotide of claim 10 to prepare an engineered regulatory T cell; and administering the engineered regulatory T cell to a subject having a non-engineered T cell, or
(ii) contacting a T cell with an unmatched donor cell: isolating a donor cell-reactive T cell; inducing FOXP3 expression in the donor-cell reactive T cell using the polynucleotide of claim 10 to prepare an engineered donor-cell reactive T cell; and administering the engineered donor-cell reactive T cell to a subject having a non-engineered T cell and an unmatched donor cell,
wherein activation of the non-engineered T cell by a T cell activating stimulus is suppressed.

24. (canceled)

25. The method of claim 23, wherein the engineered regulatory T cell and/or unmatched donor cell are human.

26. (canceled)

27. A vector comprising a polynucleotide of claim 10.

28-31. (canceled)

32. The vector of claim 27, wherein the vector is a viral vector selected from the group consisting of an adeno-associated virus vector, adenovirus vector, retrovirus vector, orthomyxovirus vector, paramyxovirus vector, papovavirus vector, picornavirus vector, lentivirus vector, herpes simplex virus vector, vaccinia virus vector, pox virus vector, and an alphavirus vector.

33. A cell comprising the vector of claim 27.

34. A pharmaceutical composition comprising a system, a vector, or a cell comprising a polynucleotide of claim 10 and a pharmaceutically acceptable carrier or excipient.

35. A method for inducing, regulating, or enhancing the expression of a FOXP3 gene in a subject comprising administering a system, a polynucleotide, a vector, a cell, or a pharmaceutical composition comprising the polynucleotide of claim 10, wherein the transcription of the FOXP3 gene (i) controls inflammation, (ii) reduces xenotransplant rejection, (iii) increases immunological tolerance to a xenotransplant, (iv) reduces allotransplant rejection, or (v) increases immunological tolerance to an allotransplant in the subject.

36-37. (canceled)

38. A method of making a genetically engineered cell, the method comprising:
(a) providing a cell, wherein the cell comprises a first nucleic acid comprising at least one targeted locus;
(b) introducing a Cas9 protein or a second nucleic acid encoding a Cas9 protein into the cell;
(c) introducing a third nucleic acid encoding at least one CRISPR guide sequence, wherein the at least one CRISPR guide sequence is configured to hybridize to the at least one targeted locus; and
(d) introducing a fourth nucleic acid into the cell, wherein the fourth nucleic acid comprises a bidirectional heterologous promoter operably linked to a nucleic acid sequence encoding a selectable marker protein and at least a first exon of a FOXP3 gene,
wherein the Cas9 protein together with the CRISPR guide sequence cleaves the first nucleic acid and a homology-directed repair of the cell introduces the fourth nucleic acid into the first nucleic acid at the at least one targeted locus and thereby genetically engineer the cell.

39. The method of claim 38, wherein the targeted locus is an intron of a FOXP3 gene.

40. The method of claim 38, wherein the selectable marker protein is a truncated nerve growth factor receptor protein.

41. (canceled)

42. A method of reducing xenotransplant rejection and/or increasing immunological tolerance to a xenotransplant in a patient, the method comprising administering to a patient prior to, at the same time as, or after transplanting a xenotransplant in the patient, a therapeutically effective amount of cells prepared according to the method of claim 3, wherein the administered cells reduce xenotransplant rejection and/or increases immunological tolerance to the xenotransplant.

43. (canceled)

44. A method of reducing allotransplant rejection and/or increasing immunological tolerance to an allotransplant in a patient, the method comprising administering to a patient prior to, or at the same time as, transplanting an allotransplant in the patient, a therapeutically effective amount of cells prepared according to the method of claim 38, wherein the administered cells reduce allotransplant rejection and/or increase immunological tolerance to the allotransplant.

45. (canceled)
